SEQUENCE LISTING (15 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 13 aa, Artificial Sequence
  Synthetic construct
KASGGTFSSYAIS

SEQ ID NO: 3 - protein, 17 aa, Artificial Sequence
  Synthetic construct
GIIPIFGTANYAQKFQG

SEQ ID NO: 4 - protein, 16 aa, Artificial Sequence
  Synthetic construct
ARSPDYSPYYYYGMDV

SEQ ID NO: 5 - protein, 13 aa, Artificial Sequence
  Synthetic construct
SGSSSNIGSNTVN

SEQ ID NO: 6 - protein, 8 aa, Artificial Sequence
  Synthetic construct
YGNSNRPS

SEQ ID NO: 7 - protein, 11 aa, Artificial Sequence
  Synthetic construct
QSYDSSLSGSV

SEQ ID NO: 8 - protein, 123 aa, Artificial Sequence
  Synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSS

SEQ ID NO: 9 - protein, 111 aa, Artificial Sequence
  Synthetic construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLG

SEQ ID NO: 10 - protein, 453 aa, Artificial Sequence
  Synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 216 aa, Artificial Sequence
  Synthetic construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 12 - DNA, 1359 bp, Artificial Sequence
  Synthetic construct
caggtccagctggtccagtcaggggccgaggtcaaaaagccagggtcatctgtcaaagtgtcttgtaaggcatccgggggcacattttccagctacgctatctcctgggtgagacaggcaccagggcagggtctggagtggatgggcggaatcattcccatcttcgggaccgccaactacgctcagaagtttcagggaagggtcactattaccgccgacaaaagcacatctactgcttatatggagctgtctagtctgaggtctgaagataccgcagtgtactattgcgcccggagtcccgactatagcccttactattactatggcatggatgtctggggccagggaaccacagtgacagtctcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 13 - DNA, 648 bp, Artificial Sequence
  Synthetic construct
cagtccgtcctgacacagccaccctcagcctctggcacccctgggcagcgagtgacaatctcttgttctgggagttcctcaaatattggtagtaacaccgtgaattggtaccagcagctgcccggcacagcacctaagctgctgatctatggaaactcaaataggccatccggagtccccgaccggttctctggtagtaaatcaggcacttccgccagcctggctattagcgggctgcagtctgaggacgaagccgattactattgccagtcttacgattccagcctgtctggaagtgtgtttggcggagggatcaagctgaccgtcctgggccagcctaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctgcagaatgctct

SEQ ID NO: 14 - protein, 443 aa, Artificial Sequence
  Synthetic construct
QVQLQQSGADLAKPGSSVKISCKASGYNFNSYYINWIKQTTGQGLEYIGYINTVSGTTKYSEKFKGKATLTVDKSSSTAFMQLSSLTPDDSAVYYCARGTIVLDDYWGQGVKVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPSKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 15 - protein, 220 aa, Artificial Sequence
  Synthetic construct
DIVMTQTPSSQAVSAGEKVTMSCKSSQSLLYNEKKKNYLAWYQQKPGQSPKLLIYWASTRESGVPDRFLGSGSGTDFTLTINSVQAEDLAVYYCQQSYDFPRTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

(19) United States
(12) Patent Application Publication
(10) Pub. No.: US 2019/0269666 A1
(43) Pub. Date: Sep. 5, 2019

(54) COMBINATION THERAPY WITH MERESTINIB AND ANTI-PD-L1 OR ANTI-PD-1 INHIBITORS FOR USE IN THE TREATMENT OF CANCER

(71) Applicant: Eli Lilly and Company, Indianapolis, IN (US)

(72) Inventors: David Arlen SCHAER, Mamaroneck, NY (US); Richard A. WALGREN, New Palestine, IN (US); Sau-Chi Betty YAN, Indianapolis, IN (US)

(21) Appl. No.: 16/320,238

(22) PCT Filed: Jul. 21, 2017
(86) PCT No.: PCT/US2017/043194

§ 371 Date: Jan. 24, 2019

Related U.S. Application Data
(60) Provisional application No. 62/368,759, filed on Jul. 29, 2016.

(51) Int. Cl. A61K 31/4439; A61P 35/00; C07K 16/28; A61K 9/00

(52) U.S. Cl. CPC A61K 31/4439; A61P 35/00; C07K 16/2818; A61K 9/0019; A61K 9/0056; C07K 16/2827

(57) ABSTRACT

The present invention relates to a combination therapy with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide (merestinib, LY2801653), or a pharmaceutically acceptable salt thereof, and antibodies that bind human co-inhibitory immune checkpoints such as Programmed Death 1 Ligand 1 (PD-L1) or Programmed Death 1 (PD-1) for the treatment of various cancers.

The present invention relates to a combination therapy with N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide (merestinib, LY2801653), or a pharmaceutically acceptable salt thereof, and antibodies that bind human co-inhibitory immune checkpoints such as Programmed Death 1 Ligand 1 (PD-L1) or Programmed Death 1 (PD-1) for the treatment of various cancers.

Tumor cells escape detection and elimination by the immune system through various mechanisms. Endogenously, immune checkpoint pathways are used in maintenance of self-tolerance and control of T cell activation. Binding of the PD-1 ligand, PD-L1 and PD-L2, to the PD-1 receptor found on T cells, inhibits T cell proliferation and cytokine production. Upregulation of PD-1 ligands occurs in some tumors and signaling through this pathway contributes to inhibition of active T-cell immune surveillance of tumors. Inhibition of PD-1 or PD-L1, have been shown to restore immune mediated destruction of tumors. Clinical research has found that targeting the PD-1 or PD-L1 with antagonist antibodies to at least one of these proteins releases the PD-1 pathway mediated inhibition of the immune response, including the anti-tumor response.

Several immune checkpoint therapies for treating cancer have recently been approved by the FDA, including anti-PD-1 antibodies (pembrolizumab/KEYTRUDA™; nivolumab/OPDIVO™), and anti-PD-L1 antibody (atezolizumab/TECENTRIQ™). These immune-oncology compounds demonstrate treatment response in multiple tumor types. However, treatment response is demonstrated only in a sub-population of patients in the approved tumor indication.

N-(3-Fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide (CAS #1206799-15-6),

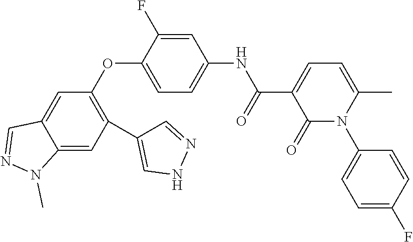 [SMILES: Cc1ccc(C(=O)Nc2ccc(Oc3cc4cnn(C)c4cc3-c3cn[nH]c3)c(F)c2)c(=O)n1-c1ccc(F)cc1]

herewith referred to as merestinib, is an ATP competitive type II kinase inhibitor that targets MET and MST1R (aka RON). In addition to MET and MST1R, merestinib also targets a few other oncokinases, including AXL, MERTK, TEK (aka Tie2), NTRK1/2/3, ROS1 and MKNK1/2 (Yan, S. B., et al., Invest New Drugs. Vol 31:833-844, 2013). These merestinib targets, when dysregulated (such as with activating mutation, amplification or with chromosomal translocation), are implicated to be tumor oncogenic drivers. Furthermore, some of these targets, such as MST1R, AXL and MERTK also can play a role in immune cell function (Eyob, H. et al., Cancer Dis. Vol 3:751-760, 2013; Lemke, G., Cold Spring Harbor Perspectives Biol. Vol 5:a009076, 2013; Lemke, G. et al., Nat Rev Immunol. Vol 8:327-336, 2008; Rothlin, C. V., Lemke, G., Curr Opin Immunol. Vol 22:740-746, 2010; Paolino, M., et al., Nature, Vol 507:508-512, 2014). Recent data from the literature also identified AXL as one of the major markers for EMT (epithelial-mesenchymal transition) in 11 different types of tumors (Mak, M. P., et al., Clin Cancer Res. Vol 22:609-620, 2016). The elevation of EMT markers in these tumor types was found to be positively correlated to increased expression of immune checkpoint markers in the tumors such as CTLA-4, PD-1, and PD-L1. Up-regulation of these immune checkpoint markers on tumors is a potential mechanism of tumor escaping immune surveillance, resulting in tumor metastasis and growth.

Broadly applicable therapies for cancer still remains elusive and, thus, there exists a need for more and different therapies that may prove to be effective in treating cancer. Merestinib may be able to synergize when combined with an immune checkpoint agent such as anti-PD-L1 or anti-PD-1 antibody to enhance and to broaden the treatment response of tumor regression in patients with different tumor types.

Combination therapies with anti-PD-L1 antibodies and MET inhibitors including merestinib have been disclosed (see WO 2016/061142). However, the present invention discloses herein methods for treating cancer, for example, lung cancer, non-small cell lung cancer (NSCLC), breast cancer, melanoma, colorectal cancer, pancreatic cancer, biliary tract cancer, melanoma (including uveal melanoma), sarcoma, bladder cancer, renal cancer, urinary tract cancers, head cancer, neck cancer, thyroid cancer, ovarian cancer, hereditary papillary renal cell carcinoma, hepatocellular carcinoma, gastric cancer and/or tumors which display alterations in MET, MSTR1 (aka RON), AXL, KRAS, FLT3, DDR1/2, ROS1, NTRK1/2/3, MKNK1/2 and its substrate EIF4E in a patient that provides enhanced and/or unexpected beneficial therapeutic effects from the combined activity of merestinib, or a pharmaceutically acceptable salt thereof, and an anti-human PD-L1 antibody or an anti-human PD-1 antibody as compared to the therapeutic effects provided by either agent alone. Furthermore, the present invention discloses methods for treating cancer, for example, lung cancer, non-small cell lung cancer (NSCLC), breast cancer, melanoma, colorectal cancer, pancreatic cancer, biliary tract cancer, sarcoma, bladder cancer, renal cancer, thyroid cancer, ovarian cancer, urinary tract cancers, melanoma (including uveal melanoma), head cancer, neck cancer, hereditary papillary renal cell carcinoma, hepatocellular carcinoma, gastric cancer and/or tumors which display alterations in MET, MSTR1 (aka RON), AXL, KRAS, FLT3, DDR1/2, ROS1, NTRK1/2/3, MKNK1/2 and its substrate EIF4E in a patient as part of a specific treatment regimen that provides enhanced and/or unexpected beneficial therapeutic effects from the combined activity of merestinib, or a pharmaceutically acceptable salt thereof, and an anti-human PD-L1 antibody or an anti-human PD-1 antibody as compared to the therapeutic effects provided by either agent alone.

Accordingly, the present invention provides a method of treating cancer in a patient, comprising administering to the patient an effective amount of merestinib, or a pharmaceutically acceptable salt thereof, in combination with an effective amount of an anti-human PD-L1 antibody or an anti-human PD-1 antibody.

The present invention also provides a combination comprising merestinib, or a pharmaceutically acceptable salt thereof, and an anti-human PD-L1 antibody or an anti-human PD-1 antibody, for simultaneous, separate, or sequential use in the treatment of cancer.

Addtionally, the present invention further provides that the anti-human PD-L1 antibody is atezolizumab, YW243.55.S70, MEDI-4736, MSB-0010718C, durvalumab, avelumab, or MDX-1105.

Also, the present invention provides that the anti-human PD-L1 antibody is LY3300054.

The present invention also provides that the anti-human PD-1 antibody is nivolumab, pembrolizumab, pidilizumab, or AMP-224.

The present invention further provides that the cancer is lung cancer, non-small cell lung cancer (NSCLC), breast cancer, melanoma, colorectal cancer, pancreatic cancer, biliary tract cancer, melanoma (including uveal melanoma), sarcoma, bladder cancer, renal cancer, urinary tract cancers, head cancer, neck cancer, thyroid cancer, ovarian cancer, hereditary papillary renal cell carcinoma, hepatocellular carcinoma, gastric cancer and/or tumors which display alterations in MET, MSTR1 (aka RON), AXL, KRAS, FLT3, DDR1/2, ROS1, NTRK1/2/3, MKNK1/2 and its substrate EIF4E. Preferably, the cancer is breast cancer, melanoma, and/or colorectal cancer. Also preferably, the cancer is breast cancer and/or colorectal cancer.

The present invention also provides a method of treating cancer in a patient, comprising administering to the patient an effective amount of merestinib, or a pharmaceutically acceptable salt thereof, in combination with an effective amount of an anti-human PD-L1 antibody or an anti-human PD-1 antibody wherein merestinib, or a pharmaceutically acceptable salt thereof, is administered at a dose of 40 mg to 120 mg once daily in a 21-day cycle or a 28-day cycle and the anti-human PD-L1 antibody or the anti-human PD-1 antibody is administered at a dose of 1 mg/kg to 10 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle. The administration, preferably, is simultaneous.

The present invention also provides a combination comprising merestinib, or a pharmaceutically acceptable salt thereof, and an anti-human PD-L1 antibody or an anti-human PD-1 antibody, for simultaneous, separate, or sequential use in the treatment of cancer wherein merestinib, or a pharmaceutically acceptable salt thereof, is administered at a dose of 40 mg to 120 mg once daily in a 21-day cycle or a 28-day cycle and the anti-human PD-L1 antibody or the anti-human PD-1 antibody is administered at a dose of 1 mg/kg to 10 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle. The use, preferably, is simultaneous.

The present invention also provides addition of either merestinib, or a pharmaceutically acceptable salt thereof, to the treatment regimen of a patient who is already being treated with an anti-human PD-L1 antibody or an anti-human PD-1 antibody.

The present invention also provides addition of either an anti-human PD-L1 antibody or an anti-human PD-1 antibody, to the treatment regimen of a patient who is already being treated with merestinib, or a pharmaceutically acceptable salt thereof.

The term “PD-1 inhibitor” means an anti-PD-1 antibody that is a fully human, or humanized IgG, optionally optimized, monoclonal antibody or small molecule inhibitor. Anti-PD-1 antibodies are preferred PD-1 inhibitors. PD-1 inhibitors include nivolumab and pembrolizumab. Nivolumab, (OPDIVO™) is also known as iMDX-1106, MDX-1106-04, ONO-4538, or BMS-936558 and has a CAS Registry Number: of 946414-94-4. Nivolumab is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Pembrolizumab, (KEYTRUDA™) (formerly lambrolizumab), also known as Merck 3745, MK-3475 or SCH-900475, is a humanized IgG4 monoclonal antibody that binds to PD-1. Pembrolizumab is disclosed in Hamid, O. et al., New England Journal of Medicine, 2013, 369(2): 134-44; WO2009/114335; and U.S. Pat. No. 8,354,509. Additional anti-PD-1 antibodies also include pidilizumab (CT-O 1) and AMP-224. Other anti-PD-1 antibodies are disclosed in U.S. Pat. No. 8,609,089; US 2010028330; and/or US 20120114649.

The term “PD-L inhibitor” means an anti-PD-L1 antibody that is a a fully human, or humanized IgG, optionally optimized, monoclonal antibody or small molecule inhibitor. Anti-PD-L1 antibodies are preferred PD-L inhibitors. PD-L1 inhibitors include YW243.55.S70, MPDL3280A, MEDI-4736, MSB-0010718C, and MDX-1105. YW243.55.S70 is an anti-PD-L1 antibody described in WO2010/077634 and US20100203056, MDPL3280A (also known as RG7446, RO5541267, and atezolizumab) is a fully humanized Fc optimized IgG1 monoclonal antibody lacking Fc effector function that binds to PD-L1. MDPL3280A and other human monoclonal antibodies to PD-L1 are disclosed in U.S. Pat. No. 7,943,743 and US 20120039906. MEDI-4736 (also known as durvalumab) is an Fc optimized antibody to PD-L1. MSB-0010718C (also known as avelumab) is a fully human IgG1 monoclonal antibody to PD-L1. MDX-1105, also known as BMS-936559, is an anti-PD-L1 antibody described in WO2007/005874.

LY3300054 is an IgG1 variant antibody lacking Fc effector function, that binds human PD-L1 (SEQ ID NO: 1), comprising a light chain (LC) and a heavy chain (HC), wherein the light chain comprises a light chain variable region (LCVR) and the heavy chain comprises a heavy chain variable region (HCVR), and wherein the LCVR comprises light chain complementarity determining regions LCDR1, LCDR2, and LCDR3 consisting of the amino acid sequences SGSSSNIGSNTVN (SEQ ID NO: 5), YGNSNRPS (SEQ ID NO: 6), and QSYDSSLSGSV (SEQ ID NO: 7), respectively, and wherein the HCVR comprises heavy chain complementarity determining regions HCDR1, HCDR2, and HCDR3 consisting of the amino acid sequences KASGGTFSSYAIS (SEQ ID NO: 2), GIIPIFGTANYAQKFQG (SEQ ID NO: 3), and ARSPDYSPYYYYGMDV (SEQ ID NO: 4), respectively.

In some embodiments of LY3300054, the antibody binds to human PD-L1, and comprises a light chain (LC) and a heavy chain (HC), wherein the light chain comprises a light chain variable region (LCVR) and the heavy chain comprises a heavy chain variable region (HCVR), wherein the LCVR has the amino acid sequence given in SEQ ID NO: 9, and the HCVR has the amino acid sequence given in SEQ ID NO: 8. In some embodiments of LY3300054, the antibody binds to human PD-L1, comprising a light chain (LC) and a heavy chain (HC), wherein the LC has an amino acid given in SEQ ID NO; 10 and the HC has the amino acid sequence given in SEQ ID NO: 11. In an embodiment, LY3300054, the antibody, comprises two light chains and two heavy chains, wherein each light chain has the amino acid sequence given in SEQ ID NO: 11, and each heavy chain has the amino acid sequence given in SEQ ID NO: 10.

As used herein, the term “patient” refers to a mammal, preferably a human.

The terms “treatment,” “treat,” and “treating,” are meant to include the full spectrum of intervention for the cancer from which the patient is suffering, such as administration of Merestinib and a PD-1 or PD-L1 inhibitor to alleviate, slow, stop, or reverse one or more of the symptoms and to delay, stop, or reverse progression of the cancer even if the cancer is not actually eliminated.

The terms “combination,” “therapeutic combination” and “pharmaceutical combination” refer to a non-fixed dose combination, optionally packaged together with instructions for combined administration where the individual therapeutic agents, merestinib, or a pharmaceutically acceptable salt or hydrate thereof, and a PD-1 or PD-L1 inhibitor may be administered independently at the same time or separately within time intervals that allow the therapeutic agents to exert a cooperative effect.

The term “simultaneous” administration means the administration of each of merestinib and a PD-1 or PD-L1 inhibitor to a patient, which can be in a single action or in concurrent or substantially concurrent actions, such as where each of merestinib and a PD-1 or PD-L1 inhibitor are administered independently at substantially the same time or separately within time intervals that allows merestinib and a PD-1 or PD-L1 inhibitor to show a cooperative therapeutic effect.

The term “separate” administration means the administration of each of merestinib and a PD-1 or PD-L1 inhibitor to a patient from non-fixed dose dosage forms simultaneously, substantially concurrently, or sequentially in any order. There may, or may not, be a specified time interval for administration of each merestinib and a PD-1 or PD-L1 inhibitor.

The term “sequential” administration means the administration of each of merestinib and a PD-1 or PD-L1 inhibitor to a patient from non-fixed (separate) dosage forms in separate actions. The two administration actions may, or may not, be linked by a specified time interval. For example, administering merestinib two days without dosing (T.I.W.) and administering a PD-1 or PD-L1 inhibitor over a specified time once every 14 to 21 days.

The phrase “in combination with” includes the simultaneous, separate, and sequential administration of each of Merestinib and a PD-1 or PD-L1 inhibitor to a cancer patient in need of treatment.

For PD-L1, anti-murine PD-L1 clone, 178G7, is used as a surrogate antibody for LY3300054. Both 178G7 and LY3300054 block PD-1 binding to PD-L1. Both 178G7 and LY3300054 block binding of PD-L1 to CD80 (aka B7-1). 178G7 competes with previously identified surrogate antibody 10F.9G2 known to block PD-L1/PD-1 interaction and is a known surrogate for anti-human antibodies in clinic (Eppihimer et al. Microcirculation 2002:9(2):133).

The HC of 178G7 is SEQ ID NO: 14, and the LC of 178G7 is SEQ ID NO: 15.

In certain embodiments, antibody provided herein for the methods of the present invention is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in a relevant antibody may be made in order to create antibody variants with certain improved properties. Additionally, in some embodiments, modifications of the oligosaccharide in a relevant antibody may be made in order to facilitate appropriate pharmaceutical compositions comprising such antibody. Furthermore, in some embodiments, modifications of the oligosaccharide in a relevant antibody may require appropriate adjustments to the antibody dosages noted herein in order to account for such modifications during its administration, although, preferably, dosage amounts provided herein are in reference to the active substance.

Antibody variants may have a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO2001/29246; US 2003/0115614; US 2002/0164328; and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

As used herein, the term “kit” refers to a package comprising at least two separate containers, wherein a first container contains merestinib, or a pharmaceutically acceptable salt thereof, and a second container contains one or more inhibitors of co-inhibitory immune checkpoint, preferably a PD-1 antibody or a PD-L1 antibody. A “kit” may also include instructions to administer all or a portion of the contents of these first and second containers to a cancer patient.

As used herein, the terms “cancer” and “cancerous” refer to or describe the physiological condition in patients that is typically characterized by unregulated cell proliferation.

As used herein, the term “primary tumor” or “primary cancer” is meant the original cancer and not a metastatic lesion located in another tissue, organ, or location in the subject's body.

As used herein, the term “effective amount” refers to the amount or dose of merestinib or a pharmaceutically acceptable salt thereof, and the amount or dose of an Inhibitor of co-inhibitory immune checkpoint, preferably an anti antibody PD-1 or an anti antibody PD-L1 which provides an effective response in the patient under diagnosis or treatment.

It is understood that a combination therapy of the present invention is carried out by administering an inhibitor of co-inhibitory immune checkpoint, preferably an antibody PD-1 or an antibody PD-L1 together with a compound that is a MET, AXL, MERTK, MKNK1/2, PDGFRA, TEK or MST1R inhibitor, or a pharmaceutically acceptable salt thereof, preferably, merestinib or a pharmaceutically acceptable salt thereof, in any manner which provides effective levels of an inhibitor of co-inhibitory immune checkpoint, preferably an antibody PD-1 or an antibody PD-L1 and a compound which is a MET or MST1R inhibitor, or a pharmaceutically acceptable salt thereof, preferably, merestinib or a pharmaceutically acceptable salt thereof, in the body.

As used herein, the term “effective response” of a patient or a patient's “responsiveness” to treatment with a combination of agents refers to the clinical or therapeutic benefit imparted to a patient upon administration of a compound which is a MET or MST1R inhibitor or a pharmaceutically acceptable salt thereof, preferably, merestinib, or a pharmaceutically acceptable salt thereof, and an Inhibitor of co-inhibitory immune checkpoint, preferably an antibody PD-1 or an antibody PD-L1.

As used herein, the term “in combination with” refers to the administration of a compound which is a MET or MST1R inhibitor or a pharmaceutically acceptable salt thereof, preferably merestinib, or a pharmaceutically acceptable salt thereof, and an inhibitor of co-inhibitory immune checkpoint, preferably an anti-PD-1 antibody or an anti-PD-L1 antibody wherein the MET or MST1R inhibitor or pharmaceutically acceptable salt thereof is administered prior to the inhibitor of co-inhibitory immune checkpoint.

Merestinib or a pharmaceutically acceptable salt thereof, is preferably formulated as a pharmaceutical composition using a pharmaceutically acceptable carrier and administered by a variety of routes. Preferably, such compositions are for oral administration. A PD-1 or PD-L1 inhibitor is preferably formulated as a pharmaceutical composition using a pharmaceutically acceptable carrier and administered by a parenteral route, preferably intravenous infusion. Preferably, such compositions may be a lyophilized powder or a liquid composition. Reconstitution or dilution to ready for administration dosages are according to label or by routine by routine skill in the art. Such pharmaceutical compositions and processes for preparing them are well known in the art. See, for example, HANDBOOK OF PHARMACEUTICAL EXCIPIENTS, 5^{th} edition, Rowe et al., Eds., Pharmaceutical Press (2006); and REMINGTON: THE SCIENCE AND PRACTICE OF PHARMACY (Troy, et al., Eds., 21^{st} edition, Lippincott Williams & Wilkins (2006).

Merestinib is capable of reaction with a number of inorganic and organic counterions to form pharmaceutically acceptable salts. Such pharmaceutically acceptable salts and common methodology for preparing them are well known in the art. See, for example, P. Stahl, et al., HANDBOOK OF PHARMACEUTICAL SALTS: PROPERTIES, SELECTION AND USE, (VCHA/Wiley-VCH, 2002); S. M. Berge, et al., “Pharmaceutical Salts,” Journal of Pharmaceutical Sciences, Vol. 66, No. 1, January 1977. A pharmaceutically acceptable salt of merestinib may require appropriate adjustments to the merestinib dosages noted herein in order to account for the salt during its administration although, preferably, dosage amounts provided herein are in reference to the active substance.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

Merestinib, or a pharmaceutically acceptable salt thereof, is administed at a dose of 40 mg to 120 mg once daily in a 21-day cycle or a 28-day cycle in combination with an anti-PD-1 antibody or an anti-PD-L1 antibody. More preferably, merestinib, or a pharmaceutically acceptable salt thereof, is administed at a dose of 80 mg once daily in a 21-day cycle or a 28-day cycle in combination with an anti-PD-1 antibody or an anti-PD-L1 antibody. Even more preferably, merestinib, or a pharmaceutically acceptable salt thereof, is administed at a dose of 120 mg once daily in a 21-day cycle or a 28-day cycle in combination with an anti-PD-1 antibody or an anti-PD-L1 antibody.

An anti-PD-1 antibody or an anti-PD-L1 antibody is administered at a dose of 1 mg/kg to 10 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. More preferably, an anti-PD-1 antibody or an anti-PD-L1 antibody is administered at a dose of 2 mg/kg to 8 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. Even more preferably, an anti-PD-1 antibody or an anti-PD-L1 antibody is administered at a dose of 5 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg.

LY3300054 is administered at a dose of 1 mg/kg to 10 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. More preferably, LY3300054 is administered at a dose of 2 mg/kg to 8 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. Even more preferably, LY3300054 is administered at a dose of 5 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg.

An anti-PD-1 antibody or an anti-PD-L1 antibody is administered at a dose of 50 mg to 1500 mg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. More preferably, an anti-PD-1 antibody or an anti-PD-L1 antibody is administered at a dose of 200 mg to 1200 mg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. Even more preferably, an anti-PD-1 antibody or an anti-PD-L1 antibody is administered at a dose of 700 mg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg.

LY3300054 is administered at a dose of 50 mg to 1500 mg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. More preferably, LY3300054 is administered at a dose of 200 mg to 1200 mg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg. Even more preferably, LY3300054 is administered at a dose of 700 mg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle in combination with merestinib, or a pharmaceutically acceptable salt thereof, administed at a dose of 40 mg to 120 mg once daily in a 21-day cycle or a 28-day cycle, more preferably at 80 mg or at 120 mg.

Treatment interuptions or administration interuptions of a short or long duration may arise at various times, for example, due to clinical scheduling delays, timing allowances for mitigating treatment emergent adverse events, remission of the relevant cancer type, and the like. Should further treatment or administration be desired or required, the present invention includes dosing regimens where the treatment or administration of merestinib along with an anti-PD-1 antibody or anti-PD-L1 antibody can be restarted or resumed.

A PD-1 or PD-L1 inhibitor may be prepared by the procedures described in U.S. Pat. No. 8,008,449 and WO2006/121168; Hamid, O. et al., New England Journal of Medicine, 2013, 369 (2): 134-44; WO2009/114335, and U.S. Pat. Nos. 8,354,509; 8,609,089, US 2010028330, and/or US 20120114649; WO2007/005874; WO2010/077634; U.S. Pat. No. 7,943,743 and US 20120039906; or by procedures well known and routinely used by one skilled in the art.

The route of administration may be varied in any way, limited by the physical properties of the drugs and the convenience of the patient and the caregiver. Preferably, an antibody PD-1 or an antibody PD-L1, preferably LY3300054, is formulated for parenteral administration, such as intravenous or subcutaneous administration. Preferably, merestinib, or a pharmaceutically acceptable salt thereof, is formulated for oral or parenteral administration, including intravenous or subcutaneous administration.

Merestinib may be formulated into a tablet or capsule. Such pharmaceutical compositions and processes for preparing same are well known in the art. (See, e.g., Remington: The Science and Practice of Pharmacy, L. V. Allen, Editor, 22^{nd} Edition, Pharmaceutical Press, 2012). For example, merestinib may be formulated into a tablet. Such tablet can be made from a composition of 20% merestinib:hydroxy propyl methyl cellulose acetate auccinate (HPMCAS) Medium Grade (M) (HPMCAS-M) Spray Dried Dispersion (SDD). The 20% merestinib:HPMCAS-M SDD is made from a spray solution composition (wt %) containing merestinib (1%), HPMCAS-M (4%) and acetone (85.5%) and purified water (9.5%). Ensure merestinib is fully solubilized in the acetone/water solution before addition of the polymer. Before initiating spray drying to make the SDD composition, visually confirm that the polymer is dissolved. The resulting SDD composition is a 20% merestinib:HPMCAS-M SDD (mg/g) with merestinib (200 mg/g) and HPMCAS-M (800 mg/g). If necessary, the amount of drug substance may be adjusted to take into account the assay of the drug substance. If required to maintain mass balance, the weight of HPMCAS-M may be adjusted according to slight changes in assay of the drug substance. Acetone and purified water are removed during processing to residual levels. The formulation composition may contain, for example, SDD merestinib and other excipients such as diluent (e.g., microcrystalline cellulose and mannitol), disintegrant (e.g., croscarmellose sodium), surfactant (e.g., sodium lauryl sulphate), glidant (e.g., syloid silicon dioxide) and/or lubricant (e.g., sodium stearyl fumarate). The making of the tablet involves spray drying to produce the SDD of merestinib followed by roller compaction and compression into tablets. The tablets are then film-coated with HPMC based color mixture.

For example, merestinib may be formulated into a tablet. Such tablet can be made from a composition of 20% merestinib:hydroxy propyl methyl cellulose acetate auccinate (HPMCAS) Medium Grade (M) (HPMCAS-M) Spray Dried Dispersion (SDD). The 20%, merestinib:HPMCAS-M SDD is made from a spray solution composition (wt %) containing merestinib (1%), HPMCAS-M (4%) and acetone (85.5%) and purified water (9.5%). Ensure merestinib is fully solubilized in the acetone/water solution before addition of the polymer. Before initiating spray drying to make the SDD composition, visually confirm that the polymer is dissolved. The resulting SDD composition is a 20% merestinib:HPMCAS-M SDD (mg/g) with merestinib (200 mg/g) and HPMCAS-M (800 mg/g). If necessary, the amount of drug substance may be adjusted to take into account the assay of the drug substance. If required to maintain mass balance, the weight of HPMCAS-M may be adjusted according to slight changes in assay of the drug substance. Acetone and purified water are removed during processing to residual levels. The formulation composition may contain, for example, SDD merestinib and other excipients such as diluent (e.g., microcrystalline cellulose and mannitol), disintegrant (e.g., croscarmellose sodium), surfactant (e.g., sodium lauryl sulphate), glidant (e.g., syloid silicon dioxide) and/or lubricant (e.g., sodium stearyl fumarate). The making of the tablet involves spray drying to produce the SDD of merestinib followed by roller compaction and compression into tablets. The tablets are then film-coated with HPMC based color mixture.

An example tablet for a unit formula of merestinib SDD film coated tablet, 40 mg dose strength, is described in Chart 1.

CHART 1
Example Unit Formula of N-(3-fluoro-4-(1-methyl-6-(1H-pyrazol-
4-yl)-1H-indazol-5-yloxy)phenyl)-1-(4-fluorophenyl)-6-methyl-2-
oxo-1,2-dihydropyridine-3-carboxamide (merestinib) Tablets 40 mg

| | | |
|---|---|---|
| Ingredient | Function | mg/Tablet |

Active Ingredient

| | | |
|---|---|---|
| 20% merestinib: 80% HPMCAS-M | Active | 200 |
| SDD¹ | | |

Intragranular Excipients²

| | | |
|---|---|---|
| Microcrystalline Cellulose (Avicel PH | Filler | 20.19 |
| 102 ®)³ | | |
| Mannitol (Pearlitol S D 200 ®) | Filler | 19.50 |
| Croscarmellose Sodium | Disintegrant | 6.50 |
| Sodium Lauryl Sulphate | Surfactant | 3.250 |
| Silicon Dioxide (Syloid 244FP ®) | Gildant | 3.250 |
| Sodium Stearyl Fumarate | Lubricant | 2.440 |

Extragranular Excipients

| | | |
|---|---|---|
| Microcrystalline Cellulose (Avicel PH | Filler | 65.00 |
| 102 ®) | | |
| Croscarmellose Sodium | Disintegrant | 3.250 |
| Sodium Stearyl Fumarate | Lubricant | 1.620 |
| Weight (mg) | Total Core Tablet | 325.0 |
| Film Coating Excipients | | |
| Color Mixture Blue 03K105008 | Film Coat | 11.38 |
| Purified Water⁴ | Suspension Vehicle | — |
| Weight (mg) | Total Coated Tablet | 336.4 |

¹If necessary, the amount of SDD will be adjusted to take into account of the assay of the dispersion.
²A reasonable variation of ±10% is allowed for each excipient unless otherwise stated.
³To accommodate changes in SDD potency and to maintain the total tablet weight, the weight of microcrystalline cellulose may be adjusted if necessary.
⁴Purified water is removed during processing to residual levels.

The antibody of the present invention, or pharmaceutical compositions comprising the same, may be administered by parenteral routes (e.g., subcutaneous and intravenous). An antibody of the present invention may be administered to a patient alone with pharmaceutically acceptable carriers, diluents, or excipients in single or multiple doses. Pharmaceutical compositions of the present invention can be prepared by methods well known in the art (e.g., Remington: The Science and Practice of Pharmacy, 22^{nd} ed. (2012), A. Loyd et al., Pharmaceutical Press) and comprise an antibody, as disclosed herein, and one or more pharmaceutically acceptable carriers, diluents, or excipients.

The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of metastatic spread without shrinkage of the primary tumor, may induce shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and/or cell cycle activity, tissue-based biomarkers for angiogenesis and/or cell cycle activity, tissue-based assessment of immune cells, and measurement of response through radiological imaging. The following Examples illustrate the activity of each of merestinib alone, a PD-1 inhibitor alone, or a PD-L1 inhibitor alone and the combination of merestinib and a PD-1 or PD-L1 inhibitor.

### Purification and Characterization of LY3300054

### Antibody Expression and Purification

The polypeptides of the variable regions of the heavy chain and light chain, the complete heavy chain and light chain amino acid sequences of LY3300054, and the nucleotide sequences encoding the same, are listed below in the section entitled “Amino Acid and Nucleotide Sequences.” In addition, the SEQ ID NOs for the light chain, heavy chain, light chain variable region, and heavy chain variable region of LY3300054 are shown in Chart 2.

The antibodies of the present invention, including, but not limited to, LY3300054 can be made and purified essentially as follows. An appropriate host cell, such as HEK 293 or CHO, can be either transiently or stably transfected with an expression system for secreting antibodies using an optimal predetermined HC:LC vector ratio or a single vector system encoding both HC and LC. Clarified media, into which the antibody has been secreted, may be purified using any of many commonly-used techniques. For example, the medium may be conveniently applied to a MabSelect column (GE Healthcare), or KappaSelect column (GE Healthcare) for Fab fragment, that has been equilibrated with a compatible buffer, such as phosphate buffered saline (pH 7.4). The column may be washed to remove nonspecific binding components. The bound antibody may be eluted, for example, by pH gradient (such as 20 mM Tris buffer pH 7 to 10 mM sodium citrate buffer pH 3.0, or phosphate buffered saline pH 7.4 to 100 mM glycine buffer pH 3.0). Antibody fractions may be detected, such as by sodium dodecyl sulfate polyacrylamide gel (SDS-PAGE), and then may be pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purity of the LY3300054 after these chromatography steps is greater than 95%. The product may be immediately frozen at −70° C. or may be lyophilized.

CHART 2
SEQ ID NOs

| | LY3300054 |
|---|---|
| HCVR | 8 |
| LCVR | 9 |
| Heavy chain | 10 |
| Light chain | 11 |

### Binding Kinetics and Affinity

The kinetics and equilibrium dissociation constant (K_{D}) for human PD-L1 is determined for antibodies of the present invention using surface plasmon resonance (Biacore).

Immobilization of antibodies of the present invention as ligand on to sensor chip surface is performed at 25° C. Soluble human PD-L1-Fc fusion protein (and in some cases, cynomolgus monkey PD-L1-Fc fusion proteins) is injected as analyte at concentrations ranging from 0.0123 nM-9 nM. The analysis is performed at 37° C. The contact time for each sample is 180 seconds at 30 μL/minute. The dissociation time was 240-1500 seconds. The immobilized surface is regenerated for 18 seconds with 0.95 M NaCl/25 mM NaOH at 30 μL/minute, and then stabilized for 30 seconds. Binding kinetics are analyzed using the Biacore T200 Evaluation software (Version 3.0). Data are referenced to a blank flow cell, and the data are fit to a 1:1 binding model.

In experiments performed essentially as described in this assay, LY3300054 binds to human PD-L1 with a K_{D} of 82 pM.

CHART 3
Binding By SPR Of LY3300054

| Binding to LY3300054 | Kon (1/Ms) | Koff (1/s) | K_{D} (pM) |
|---|---|---|---|
| Human PD-L1 | 1.40E+06 | 1.14E−04 | 82 |
| Cyno PD-L1 | 1.51E+06 | 1.84E−04 | 122 |

### ELISA Analysis: LY3300054 Binds to Recombinant PD-L1

The ability for antibodies of the present invention to bind human PD-L1 can be measured with an ELISA assay. For the PD-L1 binding assay, a 96-well plate (NUNC®) is coated with human PD-L1-Fc (R&D Systems) overnight at 4° C. Wells are blocked for 2 hours with blocking buffer (PBS containing 5% nonfat dry milk). Wells are washed three times with PBS containing 0.1% TWEEN®-20. Anti-PD-L1 antibody or control IgG (100 μL) is then added and incubated at room temperature for 1 hour. After washing, the plate is incubated with 100 μL of goat anti-human IgG F(ab′)2-HRP conjugate (Jackson Immuno Research) at room temperature for 1 hour. The plates are washed and then incubated with 100 μL of 3,3′, 5,5′-tetra-methylbenzidine. The absorbance at 450 nm is read on a microplate reader. The half maximal effective concentration (EC₅₀) is calculated using GraphPad Prism 6 software.

In experiments performed essentially as described in this assay, LY3300054 binds to human PD-L1 with an EC₅₀ of 0.11 nM. LY3300054 retains its binding activities after 4 weeks under all three temperature conditions, 4° C., 25° C. and 40° C. LY3300054 showed a similar binding activity to PD-L1 as S70 and 2.14H9OPT.

### Flow Cytometric Analysis: LY3300054 Binds to Cell Surface PD-L1

The ability for antibodies of the present invention to bind to cell surface expressed human PD-L1 can be measured with a flow cytometric assay. MDA-MB 231 cells (PD-L1-positive human breast adenocarcinoma cell line) are added to a 96 well U-bottom plate at 1.5×10⁵ cells per well in 200 μL staining buffer and incubated at 4° C. for 30 minutes. Plate are centrifuged at 1200 rpm for 5 minutes and supernatant removed. 100 μL of antibody-biotin (serially diluted by 1:4 starting from 10 μg/ml) is added. A total of 6 serial dilutions are evaluated. After incubation at 4° C. for 30 minutes, cells are washed twice with DPBS. 100 μL of detection buffer containing 5 μL streptavidin-PE is added. After incubation at 4° C. for 30 more minutes, plate is centrifuged and washed twice with DPBS. Cells are re-suspended in 200 μL DPBS for FACS analysis.

In experiments performed essentially as described in this assay, LY3300054 binds to cell surface PD-L1 on MDA-MB231 cells in a dose dependent manner with an EC₅₀ of 0.14 nM.

ELISA Analysis: LY3300054 Blocks the Interaction of PD-L1 with PD-1

The ability for antibodies of the present invention to block PD-L1 binding to PD-1 can be measured in an ELISA assay. For the receptor-ligand blocking assay, varying amounts (of anti-PD-L1 antibody or control IgG are mixed with a fixed amount of biotinylated PD-L1-Fc fusion protein (100 ng/well) and incubated at room temperature for 1 hour. The mixture is transferred to 96-well plates pre-coated with PD-1-Fc (1 μg/ml) and then incubated at room temperature for an additional 1 hour. After washing, streptavidin HRP conjugate is added, and the absorbance at 450 nm is read. IC₅₀ represents the antibody concentration required for 50% inhibition of PD-L1 binding to PD-1.

In experiments performed essentially as described in this assay, LY3300054 blocks the interaction of PD-L1 with PD-1 with an IC₅₀ of 0.95 nM. LY3300054 retains its blocking activities after 4 weeks under all three temperature conditions, 4° C., 25° C. and 40° C. LY3300054 demonstrates a similar ability to block PD-L1 interaction with PD-1 as S70 and 2.14H9OPT.

ELISA Analysis: LY3300054 Blocks the Interaction of PD-L1 with B7-1 (Aka CD80)

Human PD-L1 also binds to B7-1. The ability for antibodies of the present invention to block PD-L1 binding to B7-1 can be measured in an ELISA assay. The procedure for PD-L1/B7-1 blocking assay is similar to the PD-L1/PD-1 blocking assay, except that the plates are coated with 1 μg/ml B7-1-Fc (R&D Systems). The antibody concentration required for 50% inhibition of PD-L1 binding to PD-1 (IC₅₀) is calculated using GraphPad prism 6 software.

In experiments performed essentially as described in this assay, LY3300054 blocks the interaction of PD-L1 with B7-1 with an IC₅₀ of 2.4 nM. LY3300054 shows a similar ability to block the PD-L1 interaction with the B7-1 receptor as S70 and 2.14H9OPT.

### Evaluation of the Combination of Merestinib and Anti-PD-L1 Antibody in Preclinical Syngeneic Mouse Tumor Models

### Experimental Rational

A syngeneic mouse tumor model is used to evaluate potential combination effect of merestinib and anti-PD-L1 antibody is the CT26 colorectal cancer model using the mouse colorectal cancer line CT26. This experiment is conducted in immune competent mice (Grosso and Jure-Kunkel, Cancer Immunity Vol. 13, p. 5, January 2013; Duraiswamy et al, Cancer Research, Jun. 15, 2013 73; 3591.). Anti-PD-L1 antibody surrogate (178G7, LSN3370181) (Eppihimer et al. Microcirculation 2002:9(2):133) is used in the mouse study.

### Animals

Female BALB/c mice (18-20 gm) from Envigo (Harlan Laboratories) are used for this study. Food and water are available ad libitum. Animals are acclimated for one week prior to any experimental manipulation. The study is performed in accordance with AALAC accredited institutional guidelines.

### Compound Preparation

Merestinib is formulated as a solution in 10% PEG 400 in 90% (20% Captisol in water) once weekly. Anti-PD-L1 antibody (178G7, LSN3370181) is formulated in PBS (phosphate buffered saline).

### Study Design

CT26 cells (from internal in vivo pharmacology cell culture group lot CT26.WT.3184878) are used for implantation. Approximately 1×10⁶ cells in HBSS are implanted subcutaneously into the right hind flank of the animal under anesthesia.

Study S082015 has four groups: 1) vehicle control (10% PEG 400 in 90% [20% Captisol in water]) dosed orally once daily for 21 days; 2) single agent merestinib at 6 mg/kg once daily orally for 21 days; 3) single agent merestinib at 12 mg/kg once daily orally for 21 days; 4) single agent merestinib at 24 mg/kg once daily orally for 21 days. Dosing of merestinib is initiated at Day 6 post-implant of the CT26 cells. There are 15 animals per group. Five animals per group are taken down on Day 13 and on Day 20 post-implant of CT26 cells for the harvesting of spleens and tumors for the interrogation of the effect of merestinib on immune functions. Five animals per group remain to the end of the study (Day 26). Tumor volumes and body weight are measured bi-weekly. Tumor volume is estimated by using the formula: v=l×w2×0.536 where l=larger of measured diameter and w=smaller of perpendicular diameter. At the end of the study, animals are sacrificed using CO₂ and cervical dislocation.

Study S010516 has six groups: 1) vehicle control (10% PEG 400 in 90% [20% Captisol in water]) dosed orally once daily for 21 days and dosing initiated at Day 6 post-implant of CT26 cells; 2) single agent anti-PD-L1 antibody (178G7, LSN3370181) dosing via IP at 500 μg/animal once weekly for 3 weeks initiated at Day 6 post-implant of CT26 cells; 3) single agent merestinib at 12 mg/kg once daily orally for 21 days and initiated at Day 6 post-implant of CT26 cells; 4) concurrent combination of merestinib (12 mg/kg once daily for 21 days orally) and anti-PD-L1 antibody (500 μg/animal once weekly via IP for 3 weeks) and initiated at Day 6 post-implant of CT26 cells; 5) sequential combination of merestinib (12 mg/kg once daily for 21 days orally and initiated at Day 6 post-implant of CT26 cells) and anti-PD-L1 antibody (500 μg/animal once weekly via IP for 3 weeks and initiated at Day 13 post-implant of CT26 cells); 6) delayed concurrent combination of merestinib (12 mg/kg once daily for 21 days orally) and anti-PD-L1 antibody (500 μg/animal once weekly via IP for 3 weeks) and initiated at Day 10 post-implant of CT26 cells.

There are 15 animals per group. Five animals per group are taken down on Day 20 post-implant of CT26 cells for the harvesting of spleens and tumors for the interrogation of the effect of the combination of merestinib and anti-PD-L1 antibody combination on immune functions. Ten animals per group remain for the duration of the study unless, as pre-defined in the study protocol, the animal is to be sacrificed if the tumor volume exceeds 2500 mm³ or is ulcerated. Tumor volumes and body weight are measured bi-weekly. Tumor volume is estimated by using the formula: v=l×w2×0.536 where l=larger of measured diameter and w=smaller of perpendicular diameter.

The last dose of drug is received on Day 20, Day 27, Day 27, Day 27 and Day 31, respectively, for group 2, group 3, group 4, group 5 and group 6. On Day 64, about 1 month to 1.5 months after completing drug-dosing, animals that are still alive and with tumor response classified as PR (partial responder) or CR (complete responder) are re-challenged with a new implantation of CT26 tumor cells (1×10⁶ cells in HBSS) subcutaneously into the contralateral side of the hind flank (left side) of the animal under anesthesia. The definition of PR and CR is in the statistical method section below. Three naïve animals (study protocol S035116) are used as control and are implanted with the same batch of CT26 tumor cells (1×10⁶ cells in HBSS) subcutaneously into the left hind flank of the animal under anesthesia. Tumor growth is monitored and measured in the animals. Tumor volume is estimated by using the formula: v=l×w2×0.536 where l=larger of measured diameter and w=smaller of perpendicular diameter. Day 82 is the last day of study S010516.

At the end of the study, animals are sacrificed using CO₂ and cervical dislocation.

### Statistical Method

Tumor volume is transformed to the log scale to equalize variance across time and treatment groups. The log volume data are analyzed with a two-way repeated measures analysis of variance by time and treatment using the MIXED procedures in SAS software (Version 9.3). The correlation model for the repeated measures is spatial power. Treated groups are compared to the control group at each time point. The MIXED procedure is also used separately for each treatment group to calculate least squares means and standard errors at each time point. Both analyses account for the autocorrelation within each animal and the loss of data that occurs when animals are removed or lost before Day 33.

These data are also analyzed on Day 33 for statistical evidence of an increase in effect over additivity for the combination of two treatments. Contrast statements are used to test for an interaction effect at each time point using the two specific treatments that were combined. This is equivalent to the Bliss Independence method and assumes that tumor volumes can, in theory, reach zero, i.e., complete regression. The expected additive response (EAR) for the combination is calculated on the tumor volume scale as: response (EAR) EAR volume=V1*V2/V0, where V0, V1, and V2 are the estimated mean tumor volumes for the vehicle control, treatment 1 alone, and treatment 2 alone, respectively. If the interaction test is significant, the combination effect is declared statistically more or less than additive depending on the observed combination mean volume being less than or more than the EAR volume, respectively. The statistical conclusion is additive if the combination groups is statistically smaller than the vehicle and each single agent. Otherwise, there is no combination effect.

Ordinal logistic regression analysis is used to obtain the posterior probability of the combination treatment effect for synergy, additivity and antagonism on Day 64. Each animal is classified into one of 4 response categories, based on its Day 64 tumor volume: I) Progressive Disease (PD), II) Growth Delay (GD), III) Partial Response (PR) or IV) Complete and durable Regression (CR). CR is defined by tumor volume ≤25 mm³ at a specified time point. PR is defined as some measurement of regression occurring and is durable beyond the end of treatment or at a specified later time point, including durable stable disease. GD is defined as a clearly observable delay, or slowing of growth, that is different from vehicle but does not reach the level of stasis or regression. PD is defined as unperturbed exponential growth, similar to vehicle animals. With these categories, a proportional odds ordinal logistic regression model is defined using a 2-way analysis of variance with interaction treatment structure, such that a zero value for the interaction effect parameter corresponds to exact additivity. A range of additive activity is defined as −1.1 to +1.1 on the log odds scale for the interaction parameter, which corresponds to a 3-fold decrease or increase in the odds of being in the PD category, or, in general, of being in a lower set of categories, relative to exact additivity. Values of the interaction parameter less than −1.1 indicate synergy (defined as smaller odds of PD relative to exact additivity), and values greater than +1.1 indicate antagonism. A Bayesian analysis method is used to fit the model and obtain posterior probabilities, given the data, for the combination therapy being in the synergistic, additive, or antagonistic range. The range with the highest posterior probability is declared the most likely combination effect.

### Flow Cytometry Analysis

Spleens and tumors are collected from a subset of animals on the indicated study day(s) and processed for FACS analysis by homogenizing tissues through a tissue strainer to prepare a single cell suspension in culture medium. Single cell suspensions are then stained with fluorescently-labeled antibodies to identify immune markers including: CD3, CD4, CD8, CD11b, CD19, CD45, FoxP3, PD-L1, F4/80 and a fixable viability dye. Cells are analyzed using a BD LSR II flow cytometer. Data are collected and analyzed using Flowjo (Tree Star, Inc.) to measure the percentage of total live lymphocytes (Live, CD45+) in each tissue. Immune cell subsets are then measured as a percentage of CD45 cells, or as a percentage of the immediate parent population.

### Immuno-Landscape Analysis

Frozen tumor fragments of the samples collected on study Day 20 from 5 animals from each treatment arm (study S010516) are analyzed for gene expression of 80 markers using the Quantigene murine-80 panel (mu-80 immune panel #21681). Total RNA was isolated from lysed samples using the MAGMAX™ 96 Total RNA isolation kit (Life Technologies). Snap-frozen tumor fragments are lysed by adding 500 μL, of prepared lysis/binding buffer (MAGMAX™ 96 Total RNA isolation kit) with a single stainless steel bead (5 mm) and then homogenized on a TissueLyser (Qiagen) for 2 minutes at 25 Hz. 200 μL of lysate, along with 120 μL of 100% isopropanol and 40 μL of prepared magnetic beads mix are then transferred to a 96 deep well processing plate. A series of washes and a DNase incubation step are processed on the MAGMAX™ Express-96 Deep Well Magnetic Particle Processor (Life Technologies). RNA is eluted from the magnetic beads with 100 L of elution buffer after a final wash step. RNA concentration is determined spectrophotometrically to determine optical density at 260 and 280 nm and is then diluted to the final concentration of 25 ng/μl. Tissue expression of immune related genes is then measured using a QuantiGene® 2.0 Plex assay (Affymetrix) with a custom probe set (mu-80 immune panel #21681, Table 6). Four house-keeping-genes (HKG: Gusb, Hprt1, Ppib, Rps18) are used to normalize the gene expression data. Data are normalized and curated using the Genepattern script v28.8. Statistics and bioinformatics are used to analyze the data.

Five hundred ng of total RNA from samples being analyzed is added in duplicate to individual wells of a 96-well hybridization plate containing QuantiGene® magnetic capture beads, QuantiGene® probeset #21681, and QuantiGene® blocking reagent for a final volume of 100 μL. The hybridization plate is then sealed and incubated at 54° C. while agitated at 600 rpm for 16 hours. Samples are then transferred to a 96-well assay plate and beads are then sequentially hybridized first with 100 μl of QuantiGene® Pre-Amplifier Probe, then with QuantiGene® Amplifier Probe, and finally with QuantiGene® Label Probe (1 hour at 50° C. for each step, with 2-3 washes between each step using a magnetic capture plate). Streptavidin-conjugated R-Phycoerythrin (SAPE, Affymetrix) is added after the last hybridization step and incubated for 30 minutes at room temperature with agitation at 600 rpm. Samples are then analyzed on the FLEXMAP 3D® Luminex instrument (Luminex Corp). Beads (mRNA target) are identified with a red laser while the level of RNA is determined by measuring the mean fluorescence intensity (MFI) of Phycoerythrin with a green laser. Raw MFI data are then converted into relative gene expression for each gene (Normalized Adjusted Net MFI, background subtracted and normalized to 4 housekeeping genes), using a quality control analysis script with GenePattern (Broad Institute). This script performs the following calculations for each gene measure for each sample by calculating the “Net MFI” for each gene for each sample: (Sample MFI−background MFI of blank well). Next, the lower limit of detection (LLOD) is determined (average background MFI+3 standard deviation (SD)). The LLOD is then used to calculate the “Adjusted Net MFF” (if MFI>LLOD, then “Adjusted Net MFI”=“Net MFI”, if MFI<LLOD, then “Adjusted Net MFI”=LLOD−background). Relative gene expression is then calculated by normalizing for each gene's adjusted net MFI to the geometric mean of the MFI of selected housekeeping genes (HKG) including Hprt1, Gusb, Rps18 and Ppib, as shown in Table 6: (Sample Adjusted Net MFI/geometric mean Adjusted Net HKG MFI) multiplied by a scaling factor of 100. Fold change in gene expression for each gene per sample in each treatment group is then determined compared to the control group using the following formula: (Normalized Adjusted Net MFI Treatment sample/Mean Normalized adjusted Net MFI Control samples); fold change values <1=negative fold change, fold change values ≥1=positive fold change. For statistical analysis, the Normalized Adjusted Net MFI is converted to the Log 2 scale. Then the mean to the technical replicates are calculated for each gene, treatment, animal, and/or time. For each gene, the treatment groups are then analyzed with a one-way ANOVA to determine statistical significance. The FDR (false discovery rate) procedure is used to control the rate of false positives. The FDR procedure is calculated for each specific contrast from the ANOVA model across all genes.

### Results

CT26 CRC tumor cells harbor KRAS activating mutation G12D. Western blot analysis indicates that CT26 cells express low level of MET, but no detectible levels of phospho-MET (p-MET). CT26 cells also express high level of phospho-AXL (p-AXL) by western blot analysis and merestinib is shown to decrease p-AXL in vitro by more than 60% at a concentration of 500 nM in the mouse phospho-RTK array, but show very little effect on CT26 cell proliferation (IC50>20 μM).

### Study S082015

Single agent merestinib is shown to have significant anti-tumor effect on the CT26 syngeneic mouse tumor model by all three of the doses evaluated (Table 1) as compared to the vehicle control with the 24 mg/kg dose achieving tumor stasis. All three doses of merestinib are well tolerated as there was no significant loss in body weight. The 12 mg/kg once daily dose of merestinib is chosen to be used for the combination evaluation with anti-PD-L1 antibody in the CT26 syngeneic mouse model.

### Study S010516

In the combination study S010516, CT26 tumor cell implantation is on Day 0, and the last day of drug dosing is between Day 26 and Day 30, depending on the treatment arm. The last tumor measurement for the animals in the vehicle control group is on Day 33 as all the tumor volumes exceed 2500 mm³ and the animals in the vehicle group are sacrificed due to tumor load. Data from the anti-tumor effect of merestinib and anti-PD-L1 antibody combinations in the CT26 syngeneic mouse tumor model on Day 33 is shown in Table 2. All treatment groups are appear to be well tolerated as there was no death other than due to large tumor load. All 5 groups receiving treatment demonstrate significant anti-tumor effect as compared to the vehicle control group on Day 33. The concurrent combination treatment of merestinib and anti-PD-L1 antibody initiated on Day 6 (Group 4) demonstrates the most anti-tumor effect on Day 33 showing tumor regression with 43.5% tumor shrinkage from baseline. The anti-tumor effect of the concurrent combination (Group 4) is significantly better than either single agent alone on study Day 33 (Table 3.3). The anti-tumor effect of the concurrent combination (Group 4) is also shown to be additive by Day 33 (Table 3.1 and Table 3.2).

Tumor volume is continually monitored following cessation of drug treatment. For the merestinib as single agent group, upon completing the 21 day dosing period (last day of dosing on Day 26), tumors in all animals grew exponentially in subsequent study Days 33-40. Tumors in half of the animals in the anti-PD-L1 antibody treatment group display a response and regression by Day 33 (Table 4) and continue to regress over the next 30 days. Tumors in some of the animals in the combination treatment groups display continued regression between Day 33 and Day 64. This is evidenced in the change in response categories as shown in Table 4. In the concurrent combination group with dosing initiated on Day 6, the complete and durable regression responders (CR) increases from 3 on Day 33 to 9 by Day 64. In the sequential combination group, the complete and durable regression responders increase from 0 on Day 33 to 2 by Day 64.

As there is only comparator single agent treatment arm in Study S010516 for merestinib and for anti-PD-L1 antibody dosing initiated on Day 6, the ordinal regression analysis is conducted only for the concurrent combination of merestinib and anti-PD-L1 antibody with dosing initiated on Day 6. The posterior probability for the combination is 65% for synergy, 31% for additive and 4% for antagonistic. The interaction effect size (synergy over additivity) for the combination of merestinib and anti-PD-L1 antibody has a 5.8 fold lower median odds (90% probability of a range between 144 fold lower and 2.5 fold higher) of progressive disease than if the combination is only achieving additivity. The Bayesian ordinal regression analysis data indicates synergistic anti-tumor effect when merestinib is combined with anti-PD-L1 antibody treatment.

The sequential combination of merestinib (initiated on Day 6) and anti-PD-L1 antibody (initiated on Day 13), based on historical data suggests more anti-tumor effect than either single agent alone. Historical data indicates that upon initiation of anti-PD-L1 antibody dosing on Day 13 in the CT26 model would not result in any treatment benefit. The addition of merestinib in the sequential combination gives results in two partial responders and one complete and durable regression responder by Day 64 (Table 4).

On Day 64, the 5 animals (2 partial responders and 3 complete and durable regression responder) in the anti-PD-L1 group (Table 4) and the 9 animals (9 complete and durable regression responders) in the concurrent combination group (Table 4) are re-challenged with CT26 tumor cells (1×10⁶ cells) subcutaneously on the contralateral (left) hind flank region. Tumor growth is monitored and measured up to Day 82, the last day of the study. As shown in Table 5, naïve animals without prior treatment or previous exposure of CT26 cells implantation show rapid growth of tumors from CT26 cells resulting in a tumor volume ranging from 483-906 mm³. In all 5 animals in the anti-PD-L1 antibody group and in the 9 animals in the concurrent combination group, no tumor growth is observed (Table 5), indicating that the concurrent treatment results in immune memory and inhibition of tumor growth upon re-challenging with CT26 tumor cells. This immune memory may be important if translated clinically, suggesting that the combination treatment results in long term durable response that may translate into a long term survival benefit in a larger fraction of treated patients than anti-PD-L1 antibody treatment alone.

Treatment effect of merestinib, anti-PD-L1, and the concurrent combination on the 80-murine immune gene panel on the tumors is shown in Table 7. While merestinib alone does not induce any notable changes to the immune landscape based on the 80-murine immune gene panel selected (only perturbing 1 of the 80 immune genes measured as compared to vehicle control group that displays more than 2 fold change and p-value <0.05), anti-PD-L1 antibody treatment induces an inflammatory environment. Among the 46 immune genes that are significantly different between the anti-PD-L1 antibody group as compared to the vehicle control group, there are up-regulation of inflammatory markers such as Cd4, Ifnγ, Ccl3 and Ccl4 (Table 7). The concurrent combination treatment group significantly perturbs 56 of the 80 immune genes measured as compared to the vehicle control group. Only one gene (CDH1) in common between the merestinib treatment group and the anti-PD-L1 antibody group is significantly different from vehicle control (Table 8a). There is a significantly different gene expression pattern in the concurrent combination group as compared to the merestinib-alone group and the anti-PD-L1 antibody-alone group (Table 9).

Of interest are the 27 genes that are uniquely expressed by the concurrent treatment group and are significantly different from vehicle control (Table 8b). The gene expression pattern in the concurrent combination treatment shows an inflammatory tumor environment. There is an up-regulation of markers of tumor-infiltration lymphocytes such as CD45 (Ptprc), Cd3, Cd4, and Cd8. There is also up-regulation of markers of immune cell activation such as Ifnγ, Tnfrsf18 and its ligand Tnfsf18, Tnfrsf4 and its ligand Tnfsf4, and ligand (Cd40Ig) to Cd40 (co-stimulatory receptor on antigen presenting cells). There is also up-regulation of Itgam/Cd11b and Itgax/Cd11c markers, indicating the presence of macrophages and dendritic cells, respectively. There is also up-regulation of immunosuppressive enzymes such as Arg1, Nos2, Mpo, and Tdo2, which is likely in response to the initial inflammation signal. The down-regulation of Sele (E-selectin) suggests arrest of leukocyte rolling, while the up-regulation of ICAM1 may be indicative of activation, migration and extravasation of antigen presenting cells and inflammatory response.

The gene expression signature of the concurrent combination group further supports the synergy of the combination of these two compounds, resulting in an expanded portion of the treated animals that had durable total tumor regression and with immune memory that prevents tumor growth upon re-challenging with tumor cells.

### CONCLUSIONS

1) CT26 CRC tumor cells harbor KRAS activating mutation G12D, express low levels of Met, no detectible levels of p-Met, and high levels of p-Axl. In vitro, merestinib at 500 nM, decreases p-Axl by more than 60%, but shows very little effect on CT26 cell proliferation (IC50>20 μM).
2) Single agent merestinib shows significant anti-tumor effect in the CT26 syngeneic mouse tumor model in vivo as compared to vehicle control. It shows a dose-dependent reduction in tumor growth (6 mg/kg, 12 mg/kg, 24 mg/kg) dosed once daily orally when dosing is initiated 6 days post-implant of CT26 cells. All three doses are well tolerated.
3) The 12 mg/kg once daily dose of merestinib is selected for the combination study with anti-PD-L1 antibody. Merestinib single agent shows a significant anti-tumor effect at the end of the 21 day dosing; however, the tumors grew exponentially within a week upon dosing cessation. Anti-PD-L1 antibody alone also shows significant anti-tumor effect in the CT26 syngeneic mouse tumor model in vivo at a dose of 500 μg per animal (˜24 mg/kg) once weekly (3 doses) by IP injection when initiated 6 days post-implant of tumor cells. Tumors in a subset of the animals continued to regress after the last treatment over the next 30 days of observation
4) Concurrent combination of merestinib at 12 mg/kg once daily orally (for 21 days) and anti-PD-L1 antibody at 500 μg (˜24 mg/kg) once weekly (for 3 doses) beginning 6 days post-implant of tumor cells is found to have anti-tumor activity that is significantly improved over each single agent alone. The combination is well tolerated. The combination is found to be synergistic (more than the expected additivity effect of the two single agents) by Day 64 using Bayesian ordinal regression analysis, with a posterior probability of 65% for synergy.
5) Concurrent combination also increases the response rate of complete and durable tumor regression to 90% (9/10) as compared to 30% (3/10) in the anti-PD-1 antibody-alone group by study Day 64. This concurrent combination also results in “immune memory” and prevents tumor growth in the 9 complete responder mice when re-challenged with new implantation of CT26 tumor cells.
6) There is no significant change in immune cells from animals treated with merestinib aloneor with anti-PD-L1 antibody alone as compared to the concurrent combination or with vehicle control as evaluated by flow cytometry.
7) Merestinib treatment alone shows no significant change in 79 of an 80-immune gene panel (mRNA expression) in the tumors as compared to vehicle control. The 80-immune gene expression signature of the concurrent combination group differs significantly from each single agent alone.
8) Data from this preclinical study strongly supports a clinical combination of merestinib and the immune checkpoint targeting PD-L1 (anti-PD-L1 antibody) to increase the fraction of treatment responders and to prolong survival in patients with cancer.

TABLE 1
Study S 082015 - Tumor volume and body weightanalysis reported
on Day 26 as compared to the vehicle control group

| | | Merestinib | Merestinib | Merestinib |
|---|---|---|---|---|
| | Vehicle | 6 mg/kg dose | 12 mg/kg dose | 24 mg/kg dose |
| \hat{ }T/C % | NA | 52.8 | 34.8 | 13.4 |
| p-value* | NA | 0.043 | <0.001 | <0.001 |

\hat{ }T/C is tumor volume of the treated group/tumor volume of the vehicle control group
*Statistically significant (p < 0.05)
NA—not applicable

| | | Merestinib | Merestinib | Merestinib |
|---|---|---|---|---|
| | Vehicle | 6 mg/kg dose | 12 mg/kg dose | 24 mg/kg dose |
| \hat{ }Body weight change | 11.6 | 5.7 | 3.4 | 3.0 |
| from baseline (%) | | | | |
| p-value* | NA | 0.026 | 0.014 | 0.007 |

\hat{ }Much more body weight increase in the vehicle group is due to tumor burden
*Statistically significant (p < 0.05)
NA—not applicable

TABLE 2
Study S 010516 - Tumor volume analysis reported on Day 33 of
the treatment groups as compared to the vehicle control group

| | | | | Tumor | |
|---|---|---|---|---|---|
| | Mean Tumor Volume | | \hat{ }T/C | Regression | |
| Treatment Group | mm³ (SE) | | (%) | (%) | p-value* |
| Vehicle control | 5018.0 | (971.6) | NA | NA | NA |
| Anti-PD-L1 Ab alone | 198.2 | (87.2) | 2.7 | NA | <0.001 |
| Merestinib alone | 1609.0 | (286.3) | 31.2 | NA | 0.008 |
| Concurrent combination | 36.8 | (9.0) | NA | −43.5 | <0.001 |
| (dosing initiated on Day 6) | | | | | |
| Sequential combination | 342.5 | (142.6) | 5.4 | NA | <0.001 |
| Delayed concurrent combination | 663.6 | (192.5) | 11.9 | NA | <0.001 |
| (dosing initiated on Day 10) | | | | | |

\hat{ }T/C is tumor volume of the treated group/tumor volume of the vehicle control group; grand mean of all groups from baseline at Day 15 was used to compute & change of T/C or for Regression
*Statistically significant (p < 0.05)
NA—not applicable

TABLE 3.1
On Study Day 33, Interaction Tests-Tumor Volume for merestinib and anti-PD-L1
antibody indicates the concurrent combination (Initiated on Day 6) is additive

| | Mean | Mean | Mean | Mean | Expected if | | | |
|---|---|---|---|---|---|---|---|---|
| | Tumor | Tumor | Tumor | Tumor | Additive | Estimate | | |
| | Volume | Volume | Volume | Volume | Tumor | (log | | p- |
| Day | Vehicle | (PD-L1) | (Merestinib) | (Combination) | Volume | scale) | s.e. | value |
| 8 | 76.0 | 63.5 | 64.7 | 65.2 | 54.0 | 0.041 | 0.095 | 0.669 |
| 12 | 166.4 | 133.7 | 132.5 | 89.8 | 106.5 | −0.037 | 0.095 | 0.697 |
| 15 | 317.3 | 190.9 | 206.6 | 99.1 | 124.3 | −0.049 | 0.095 | 0.605 |
| 19 | 664.8 | 285.4 | 357.7 | 88.1 | 153.6 | −0.121 | 0.095 | 0.206 |
| 22 | 1042.2 | 316.3 | 491.5 | 84.4 | 149.1 | −0.124 | 0.098 | 0.211 |
| 25 | 1876.7 | 290.1 | 717.0 | 72.8 | 110.8 | −0.091 | 0.101 | 0.369 |
| 28 | 2842.4 | 247.4 | 845.3 | 56.3 | 73.6 | −0.058 | 0.106 | 0.585 |
| 33 | 5435.7 | 188.4 | 1608.2 | 36.9 | 55.8 | −0.090 | 0.112 | 0.427 |

TABLE 3.2
On Study Day 33, Interaction Tests-Tumor Volume for
merestinib and anti-PD-L1 antibody indicates the concurrent
combination (Initiated on Day 6) is additive

| | % | % | % | Expected if Additive |
|---|---|---|---|---|
| | Response^{a} | Response^{a} | Response^{a} | % Response^{a} |
| | Anti-PD-L1 Ab | Merestinib | Concurrent | Concurrent |
| Day | alone | alone | combination | combination |
| 8 | −66.2 | −65.5 | −65.3 | −71.2 |
| 12 | −28.7 | −29.4 | −52.1 | −43.2 |
| 15 | 2.5 | 14.6 | −47.2 | −33.8 |
| 19 | 20.5 | 53.7 | −53.1 | −18.1 |
| 22 | 15.1 | 35.6 | −55.0 | −20.5 |
| 25 | 6.1 | 31.3 | −61.2 | −40.9 |
| 28 | 2.3 | 24.8 | −70.0 | −60.8 |
| 33 | 0.0 | 27.1 | −80.3 | −70.3 |

^{a}% Response is % ΔT/C for tumor volumes above baseline and % Regression for tumor volumes below baseline

TABLE 3.3
Treatment comparison (vehicle, anti-PD-L1 antibody alone, merestinib
alone and concurrent combination) - log scale on Day 33

| Day | Treatinent 1 | Treatment 2 | Difference^{b} | s.e. | p-value* |
|---|---|---|---|---|---|
| 33 | Vehicle control | Anti-PD-L1 antibody | 1.460 | 0.1661 | <0.001 |
| 33 | Vehicle control | Merestinib | 0.529 | 0.1666 | 0.002 |
| 33 | Vehicle control | Concurrent | 2.168 | 0.1651 | <0.001 |
| | | combination | | | |
| 33 | Anti-PD-L1 | Concurrent | 0.708 | 0.1512 | <0.001 |
| | antibody | combination | | | |
| 33 | Merestinib | Concurrent | 1.639 | 0.1518 | <0.001 |
| | | combination | | | |

*Statistically significant (p < 0.05)
^{b}Difference = Treatment 1 − Treatment 2

TABLE 4
Treatment responders by Day 33 and by Day
64 by treatment group (10 animals/group)

| | Day 33 Treatment | | | | Day 64 Treatment | | | |
|---|---|---|---|---|---|---|---|---|
| | Responders^{c} | | | | Responders^{c} | | | |
| Treatment Group | PD | GD | PR | CR | PD | GD | PR | CR |
| Vehicle control | 10 | 0 | 0 | 0 | 10 | 0 | 0 | 0 |
| Anti-PD-L1 Ab alone | 2 | 3 | 4 | 1 | 2 | 3 | 2 | 3 |
| Merestinib alone | 9 | 1 | 0 | 0 | 9 | 1 | 0 | 0 |
| Concurrent | 1 | 0 | 6 | 3 | 1 | 0 | 0 | 9 |
| combination | | | | | | | | |
| Sequential combination | 4 | 3 | 3 | 0 | 4 | 3 | 1 | 2 |
| Delayed concurrent | 4 | 4 | 2 | 0 | 4 | 4 | 2 | 0 |
| combination | | | | | | | | |

^{c}Progressive Disease (PD), Growth Delay (GD), Partial Response (PR), Complete and durable Regression (CR). CR is defined by tumor volume ≤25 mm³ at a specified time point. PR is defined as some regression occurs and is durable beyond the end of treatment or at a specified later time point. This would also include durable stable disease. GD is defined as clearly observable delay, or slowing of growth, that is different from vehicle but does not reach the level of stasis or regression. PD is defined as unperturbed exponential growth, similar to vehicle animals.

TABLE 5
Tumor volume on Day 82 in animals after re-
challenged with CT26 tumor cells on Day 64

| Individual animal by Treatment group | Tumor Volume (mm³) |
|---|---|
| Vehicle Control group* (3 animals) | 482.7 |
| | 721.1 |
| | 905.9 |
| Anti-PD-L1 antibody group (5 animals) | 7.5 |
| | 0 |
| | 0 |
| | 0 |
| | 0 |
| Concurrent combination group (9 animals) | 0 |
| | 0 |
| | 0 |
| | 0 |
| | 0 |
| | 0 |
| | 0 |
| | 0 |
| | 0 |

*Naïve animals without prior implantation of CT26 cells (study S035116) and implanted with the same batch of CT26 cells as for the re-challenging in study S010516). Tumor cells implanted same day as Day 64 and measured on the same day as Day 82.

TABLE 6
A list of the genes in the mu-80 immune gene panel

| Cell type-specific markers | Coinhibitory/Costimulatory | Cytokines/Chemokines |
|---|---|---|
| Cd14 (macrophage, DC) | 4632428N05Rik (Vista) | Ccl2 (MCP-1) |
| Cd3e | Cd200r1 | Ccl3 (MIP-1a) |
| Cd4 | Cd274 (PD-L1) | Ccl4 (MIP-1b) |
| Cd68 (monocyte) | Cd40lg | Ccl5 (RANTES) |
| Cd8b1 | Cd86 | Cxcl1 |
| Foxp3 (regulatory T cell) | Havcr2 (TIM3) | Ifna1 |
| Itgam (Cd11b, | Icos | Ifnb1 |
| macrophage) | Lag3 | Il10 (CS1F) |
| Itgax (Cd11c, DC) | Pdcd1 (PD-1) | Il13 |
| Klrk1 (NK cell) | Pdcd1lg2 (PD-L2) | Il17a |
| Ms4a1 (B cell) | Pvr | Il2 |
| Ptprc (CD45) | Pvrl2 | II4 |
| Timd4 (Tim4) (APC) | Tigit | II5 |
| | Tnfrsf18 (GITR) | Il6 |
| | Tnfrsf4 (OXO40) | II9 |
| | Tnfrsf9 (4-1BB, CD137) | Tnf |
| | Tnfsf18 (GITRL) | |
| | Tnfsf4 (OXO40L) | |
| | Tnfsf9 (Cd137L, 4-1BBL) | |
| Immunosuppressive | | Angiogenesis/endothelial |
| enzymes | Markers of T cell activation | activation |
| Arg1 | Cd69 | Cdh5 |
| Ido1 | Gzmb | Hif1a |
| Mpo | Ifnγ | Vcam1 |
| Nos2 | | Vegfa |
| Tdo2 | | Vegfc |
| | | Icam1 (Cd54) |
| | | Sele |
| EMT markers | TGFβ pathway | House-keeping genes |
| Cdh1 | Smad4 | Gus6 |
| Epcam | Tgfb1 | Hprt1 |
| Fap | Tgfb2 | Ppib |
| Snai1 | Tgfb3 | Rps18 |
| Twist1 | Tgfbr1 | |
| Vim | Tgfbr2 | |
| Cspg4 | | |
| Co-inhibitory | Co-stimulatory | |
| 4632428n05rik (Vista) | Cd40lg | |
| Cd200r1 | Cd86 | |
| Cd274 (PD-L1) | Icos | |
| Havcr2 (Tim3) | Tnfrsf4 (OXO40) | |
| Lag3 | Tnfrsf9 (4-1BB, CD137) | |
| Pdcd1 (PD-1) | Tnfsf18 (GITRL) | |
| Pdcd1lg2 (PD-L2) | Tnfsrf18 (GITR) | |
| Pvrl2 | Tnfsf4 (OXO40L) | |
| Tigit | Tnfsf9 (CD137L, 4-1BBL) | |
| Pvr (when bound to Tigit) | Pvr (when bound to CD226) | |

TABLE 7
Differential analysis of the fold change of immune genes comparing merestinib, anti-PD-L1
antibody or the concurrent combination to the vehicle control
All the genes shown below are significantly different from
vehicle control (p ≤ 0.05) from 1-way ANOVA.

| | | | | Concurrent combination | |
|---|---|---|---|---|---|
| Merestinib alone | | Anti-PD-L1 antibody alone | | | Log₂ (difference |
| | Log₂ (difference | | Log₂ (difference | | of concurrent |
| | of merestinib to | | of anti-PD-L1 to | | combination to |
| Gene | vehicle control) | Gene | vehicle control) | Gene | vehicle control) |
| Cdh1 | 1.0 | Arg1 | 2.0 | Arg1 | 2.3 |
| Tnfrsf | 0.8 | Ccl2 | 0.6 | Ccl4 | 1.5 |
| | | Ccl3 | 2.1 | Ccl5 | 2.9 |
| | | Ccl4 | 1.7 | Cd14 | 0.4 |
| | | Ccl5 | 1.5 | Cd200rl | 0.6 |
| | | Cd14 | 0.4 | Cd274 | 2.9 |
| | | Cd200rl | 0.8 | Cd3e | 1.9 |
| | | Cd274 | 2.0 | Cd4 | 2.2 |
| | | Cd4 | 1.3 | Cd40lg | 1.2 |
| | | Cd40lg | 0.9 | Cd68 | 1.0 |
| | | Cd68 | 0.9 | Cd86 | 1.4 |
| | | Cd86 | 0.8 | Cd8b1 | 1.7 |
| | | Cdh1 | 1.0 | Cdh1 | 1.0 |
| | | Cxcl1 | 2.3 | Cspg4 | 1.0 |
| | | Epcam | 0.9 | Epcam | 1.1 |
| | | Fap | 1.1 | FAap | 2.0 |
| | | Havcr2 | 1.0 | Foxp3 | 1.7 |
| | | Hif1a | 0.8 | Havcr2 | 0.9 |
| | | Icam1 | 1.2 | Hif1a | 1.0 |
| | | Icos | 1.1 | Icam1 | 1.5 |
| | | Ifna2 | 0.9 | Icos | 1.9 |
| | | Ifng | 1.2 | Ifna2 | 0.8 |
| | | Il10 | 1.2 | Ifng | 1.5 |
| | | Il13 | 0.9 | Il10 | 1.4 |
| | | Il17a | 0.8 | Il13 | 1.1 |
| | | Il4 | 0.9 | Il17a | 1.1 |
| | | Il5 | 1.0 | Il4 | 1.1 |
| | | Il6 | 1.2 | Il5 | 0.9 |
| | | IL9 | 1.2 | Il9 | 1.1 |
| | | Itgax | 0.9 | Itgam | 1.3 |
| | | Mpo | 0.9 | Itgax | 1.4 |
| | | Ms4a1 | 0.9 | Lag3 | 1.5 |
| | | Nos2 | 3.6 | Mpo | 1.1 |
| | | Pdcd1lg2 | 2.0 | Ms4a1 | 1.0 |
| | | Pvr | 0.8 | Nos2 | 4.1 |
| | | Pvrl2 | 0.6 | Pdcd1 | 1.5 |
| | | Tdo2 | 0.9 | Pdcd1lg2 | 4.2 |
| | | Tgfb2 | 0.9 | Ptprc | 0.9 |
| | | Tgfbr2 | 0.8 | Pvrl2 | 0.7 |
| | | Timd4 | 1.4 | Rps18 | −0.8 |
| | | Tnfrsf9 | 1.3 | Sele | −1.9 |
| | | Tnfsf18 | 0.8 | Tdo2 | 1.1 |
| | | Tnfsf4 | 1.1 | Tgfb1 | 0.3 |
| | | Vegfa | 1.1 | Tgfb2 | 1.1 |
| | | Vegfc | 1.7 | Tgfbr2 | 1.6 |
| | | Vista | 1.1 | Tigit | 0.5 |
| | | | | Timd4 | 2.0 |
| | | | | Tnf | 1.6 |
| | | | | Tnfrsf18 | 1.7 |
| | | | | Tnfrsf4 | 1.3 |
| | | | | Tnfsf18 | 1.0 |
| | | | | Tnfsf4 | 0.9 |
| | | | | Twist1 | −1.4 |
| | | | | Vcam1 | −0.9 |
| | | | | Vegfc | 2.2 |
| | | | | Vista | 2.1 |

TABLE 8a
Immune gene expression in common between the merestinib treatment
alone and anti-PD-L1 antibody alone that was significantly
different from vehicle control
All the genes shown below are significantly different from
each single agent alone (p ≤ 0.05) from 1-way ANOVA.

| | | Log₂ (difference | Log₂ (difference |
|---|---|---|---|
| | | of anti-PD-L1 antibody | of merestinib |
| Gene | Annotation | to vehicle control) | to vehicle control) |
| CDH1 | EMT | 1 | 1.01 |

TABLE 8b
Immune gene expression unique to the concurrent combination
group that was significantly different from vehicle control
All the genes shown below are significantly different from
each single agent alone (p ≤ 0.05) from 1-way ANOVA.

| | | Log₂ (difference |
|---|---|---|
| | | of Concurrent |
| | | combination to vehicle |
| Gene | Annotation | control) |
| Cd3e | Cell type specific marker | 1.9 |
| Cd40lg | Co-inhibitory/co-stimulatory | 1.2 |
| Cd68 | Cell type specific marker | 1.0 |
| Cd86 | Co-inhibitory/co-stimulatory | 1.4 |
| Cd8b1 | Cell type specific marker | 1.7 |
| Cspg4 | EMT | 1.0 |
| Epcam | EMT | 1.1 |
| Foxp3 | Cell type specific marker | 1.7 |
| Il13 | Cytokine/chemokine | 1.1 |
| Il17a | Cytokine/chemokine | 1.1 |
| Il4 | Cytokine/chemokine | 1.1 |
| Itgam | Cell type specific marker | 1.3 |
| Itgax | Cell type specific marker | 1.4 |
| Lag3 | Co-inhibitory/co-stimulatory | 1.4 |
| Mpo | Immunosuppressive enzyme | 1.1 |
| Ms4a1 | Cell type specific marker | 1.0 |
| Pdcd1 | Co-inhibitory/co-stimulatory | 1.5 |
| Sele | T cell activation marker | −1.9 |
| Tdo2 | Immunosuppressive enzyme | 1.1 |
| Tgfb2 | TGFb pathway | 1.1 |
| Tgfbr2 | TGFb pathway | 1.1 |
| Timd4 | Co-inhibitory/co-stimulatory | 2.0 |
| Tnf | Cytokine/chemokine | 1.6 |
| Tnfrsf18 | Co-inhibitory/co-stimulatory | 1.7 |
| Tnfrsf4 | Co-inhibitory/co-stimulatory | 1.3 |
| Tnfsf18 | Co-inhibitory/co-stimulatory | 1.0 |
| Twist1 | EMT | −1.4 |

TABLE 9
Differential analysis of the fold change of the concurrent combination
treatment (merestinib + anti-PD-L1) as compared to each single agent
All the genes shown below are significantly different from
each single agent alone (p ≤ 0.05) from 1-way ANOVA.

| Concurrent combination | | Concurrent combination | |
|---|---|---|---|
| Vs Merestinib | | Vs anti-PD-L1 antibody | |
| | Log₂ (difference | | Log₂ (difference |
| | of concurrent | | of concurrent |
| | combination | | combination |
| Gene | to merestinib) | Gene | to anti-PD-L1 antibody) |
| Arg1 | 2.1 | Ccl2 | −0.5 |
| Ccl4 | 1.1 | Cxcl1 | −2.0 |
| Ccl5 | 1.9 | Il6 | −1.2 |
| Cd200rl | 0.7 | Itgam | 0.8 |
| Cd274 | 1.6 | Pdcd1lg2 | 2.2 |
| Cd3e | 1.4 | Pvr | −1.0 |
| Cd4 | 1.4 | Sele | −1.8 |
| Cd40lg | 0.7 | Tgfbr2 | 0.9 |
| Cd68 | 1.1 | Tnf | 1.2 |
| Cd86 | 1.0 | Tnfrsf18 | 1.3 |
| CD8b1 | 1.2 | Tnf | 1.2 |
| Fap | 2.0 | Tnfrsf18 | 1.3 |
| Foxp3 | 1.1 | Twist1 | −1.3 |
| Havcr2 | 0.7 | Vcam1 | −1.1 |
| Hof1a | 0.5 | Vista | 1.0 |
| Icam1 | 1.3 | | |
| Icos | 1.0 | | |
| Il10 | 0.9 | | |
| Il13 | 0.7 | | |
| Il17a | 0.7 | | |
| Il4 | 0.7 | | |
| Il9 | 0.7 | | |
| Itgam | 1.3 | | |
| Itgax | 1.0 | | |
| Mpo | 0.7 | | |
| Nos2 | 2.4 | | |
| Pdcd1ig2 | 3.0 | | |
| Sele | −1.8 | | |
| Tdo2 | 0.7 | | |
| Tgfb1 | 0.3 | | |
| Tgfb2 | 0.7 | | |
| Tgfbr2 | 1.6 | | |
| Timd4 | 1.7 | | |
| Tnf | 1.1 | | |
| Tnfrsf18 | 0.9 | | |
| Tnfrsf4 | 1.0 | | |
| Tnfsf18 | 0.7 | | |
| Twist1 | −1.5 | | |
| Vcam1 | −1.0 | | |
| Vegfc | 1.4 | | |
| Vista | 1.9 | | |

### In Vivo Evaluation of the Combination of Merestinib (LY2801653) with Anti-PD-L1 Antibody in the EMT6 Syngeneic Mouse Mammary Carcinoma Tumor Model

### Experimental Rationale

A syngeneic mouse tumor model is used to evaluate potential combination effect of merestinib and anti-PD-L1 antibody in the EMT6 model (Rockwell, S C, Kallman, R F, Fajardo, L F. Characteristics of a serially transplanted mouse mammary tumor and its tissue-culture-adpated derivative. J Nat Cancer Institute. 1972, 49:735-749). Anti-PD-L1 antibody surrogate (178G7, LSN3370181) (Eppihimer et al. Microcirculation 2002:9(2):133) is used in the mouse study.

### Animals

Female BALB/c mice (18-20 gm) from Envigo (Harlan Laboratories) are used for this study. Food and water are available ad libitum. Animals are acclimated for at least one week prior to any experimental manipulation. The study is performed in accordance with AALAC accredited institutional guidelines.

### Compound Preparation

Merestinib is formulated as a solution in 10% PEG 400 in 90% (20% Captisol in H₂O) once weekly. Anti-PD-L1 antibody (LSN3370181) is formulated in PBS (phosphate buffered saline).

### Study Design

EMT6 cells (from internal in vivo pharmacology cell culture group lot EMT6-3184882) are used for implantation. Approximately 5×10⁵ cells in HBSS are implanted subcutaneously into the right hind flank of the animal under anesthesia. Prior to commencing drug administration, animals are randomized to treatment groups based on body weight.

There are 4 arms in this study with 15 animals per group: 1) vehicle control; 2) merestinib 12 mg/kg QD×21, PO; 3) LSN3370181 (anti-PD-L1) 0.5 mg/mouse once weekly for 3 weeks via IP; 4) combination merestinib+anti-PD-L1 antibody; combination therapies follow the same dosing schedule as the monotherapies. Treatment is initiated on Day 6 post tumor cell implantation and continued for 21 days (final dose administered on Day 26). Tumor volumes and body weight are measured bi-weekly. Tumor volume is estimated by using the formula: v=l×w²×0.536 where l=larger of measured diameter and w=smaller of perpendicular diameter. Animals are sacrificed using CO₂ and cervical dislocation.

On Day 20 post tumor cell implant, 5 animals from each group are taken down for harvesting of spleens and tumors for mechanism of action (MOA) studies (FACs analysis, gene expression). The remaining animals continue with treatment as described above. At the end of the dosing period (Day 27 post tumor cell implant), animals are observed for tumor growth for 43 days. Animals are removed from study when tumor burden reached or exceeded 2500 mm³. To assess immune memory, on Day 70 post tumor cell implant, animals that remained on study (complete responders and partial responders) are rechallenged with new implant of EMT6 cells on the contralateral side and observed for tumor growth for 15 days.

### Statistical Method

Tumor volume is transformed to the log scale to equalize variance across time and treatment groups. The log volume data are analyzed with a two-way repeated measures analysis of variance by time and treatment using the MIXED procedures in SAS software (Version 9.3). The correlation model for the repeated measures is spatial power. Treated groups are compared to the control group at each time point. The MIXED procedure is also used separately for each treatment group to calculate least squares means and standard errors at each time point. Both analyses account for the autocorrelation within each animal and the loss of data that occurs when animals are removed or lost before end of study.

Bayesian ordinal logistic regression analysis is used to obtain the posterior probability of the combination treatment effect for synergy, additivity or antagonism was conducted on Day 69 for this study. Each animal was classified into one of 3 response categories, based on its Day 69 tumor volume and by comparison to the vehicle data: I) Non-responder (NR), II) Partial Responder (PR), III) Complete Responder (CR). CR is defined by tumor volume 525 mm³ at Day 69. NR is defined as unperturbed exponential growth, similar to vehicle animals. At least two time points with tumor volume below the vehicle mean minus 3 SD were required to classify an animal as a PR. With these categories, a proportional odds ordinal logistic regression model is fitted using a 2-way analysis of variance with interaction treatment structure, such that a zero value for the interaction effect parameter corresponded to exact additivity. A range of additive activity was defined as −1.1 to +1.1 on the log odds scale for the interaction parameter, which corresponds to a 3-fold decrease or increase in the odds of being in the NR category, or in general of being in a lower set of categories, relative to exact additivity. Values of the interaction parameter less than −1.1 indicate synergy (smaller odds of NR relative to exact additivity), and values greater than +1.1 indicate antagonism. A Bayesian analysis method was used to fit the model and obtain posterior probabilities, given the data, for the combination therapy being in the synergistic, additive, or antagonistic range. The range with the highest posterior probability was declared the most likely combination effect.

### Results

Merestinib (LY2801653) (12 mg/kg) demonstrated weak single agent anti-tumor activity in the EMT6 syngeneic mouse mammary carcinoma tumor model. While tumor growth delay was observed during treatment (Table 1), once merestinib administration ended the tumors grew quickly (Table 1). The last day of treatment was Day 26. Data shown in Table 1 were on study Day 31. Four of the animals in the vehicle group were removed by Day 31 due to tumor burden (tumor volume ≥2500 mm³). The tumor volume of the merestinib treated group on Day 31 was T/C=35.2% as compared to the control group (Table 1). By study Day 49, all animals in the merestinib treated group were removed due to tumor burden (tumor volume ≥2500 mm³). The anti-PD-L1 treatment group and the anti-PD-L1 combination with merestinib treatment group showed tumor regression by Day 31 (Table 1), more so in the combination treatment group. The tumors in some of the animals in the two treatment groups continued to regress over the next 38 days to Day 69.

Anti-PD-L1 antibody (LSN3370181) was shown to have potent and durable anti-tumor activity in this model as a single agent with 6/10 complete responders at the end of study (Day 69) (Table 2). Combining merestinib with anti-PD-L1 resulted in greater efficacy as compared to either single agent with 9/10 of the animals being complete responders by end of the study (Day 69) (Table 2). The Bayesian Ordinal Logistic Regression analysis conducted for the combination of merestinib and anti-PD-L1 antibody determined the posterior probability for the combination to be 78% for synergy, 19% for additive and 3% for antagonistic. Therefore, the combination of merestinib with anti-PD-L1 antibody results in a synergistic anti-tumor effect.

On Day 70 post implant, all animals classified as complete responders and several partial responders were rechallenged on the contralateral side with EMT6 cells to assess immune memory. Table 3 provides measurements of the primary tumor at the time of rechallenge and at 2 additional timepoints following rechallenge. Measurements of the secondary tumor are also provided in the table. Animals classified as complete responders resisted rechallenge indicating an induction of immune memory, while the 3 animals classified as partial responders had observed tumor growth at both the primary and secondary sites.

Treatments were well tolerated with no significant clinically meaningful weight loss relative to the control group (Data not shown).

TABLE 10
Tumor volume analysis reported on Day 31 comparing
treatment groups to the vehicle control group

| Group | N | T/C (%) | Regression (%) | p-value |
|---|---|---|---|---|
| Vehicle | 6/10 | NA | NA | NA |
| merestinib | 10/10 | 35.2 | | 0.124 |
| PD-L1 | 10/10 | | 34.8 | <0.001 |
| merestinib/PD-L1 | 10/10 | | 69.8 | <0.001 |

The data in Table 10 show that more tumor regression was observed in the combination treatment group than the anti-PD-L1 treatment group. No tumor regression was observed in the merestinib treatment group.

TABLE 11
Treatment responders by Day 69 by treatment group (10 animals/group)
for the Bayesian ordinal logistic regression analysis

| | | Day 69 Treatment Responders^{c} | | |
|---|---|---|---|---|
| Group # | Treatment Group | NR | PR | CR |
| 1 | Vehicle | 10 | 0 | 0 |
| 2 | Merestinib | 10 | 0 | 0 |
| 3 | Anti-PD-L1 antibody | 2 | 2 | 6 |
| 4 | Merestinib + PD-L1 Ab | 0 | 1 | 9 |

^{c}Non-responder (NR), Partial Responder (PR), Complete Responder (CR). CR is defined by tumor volume ≤25 mm³ at Day 69. NR was defined as unperturbed exponential growth, similar to vehicle animals. At least two time points with tumor volume below the vehicle mean minus 3 SD were required to classify an animal as a PR

The data of Table 11 show that the combination of merestinib with anti-PD-L1 resulted in greater efficacy as compared to either single agent. Each animal is classified to the response to treatment as one of three categories. The response data is used for the Bayesian ordinal logistic regression analysis which shows that the combination treatment is synergistic.

TABLE 12
Re-challenged tumor volumes in EMT6 model

| | | Tumor Volume (mm³) | | | | | |
|---|---|---|---|---|---|---|---|
| Treatment | Animal | Study Day 69 | | Study Day 78 | | Study Day 84 | |
| group | ID | Right | Left | Right | Left | Right | Left |
| PD-L1 Ab | 3-06 | 0 | — | 0 | 39 | 0 | 0 |
| (Group 3) | 3-10 | 0 | — | 0 | 54 | 0 | 0 |
| | 3-11 | 0 | — | 0 | 26 | 0 | 0 |
| | 3-12 | 0 | — | 0 | 46 | 0 | 0 |
| | 3-13 | 388 | — | 847 | 123 | 1173 | 293 |
| | 3-14 | 0 | — | 0 | 40 | 0 | 0 |
| | 3-15 | 0 | — | 0 | 25 | 0 | 6 |
| Merestinib + | 4-06 | 0 | — | 0 | 66 | 0 | 0 |
| PD-L1 Ab | 4-07 | 0 | — | 0 | 18 | 0 | 0 |
| (Group 4) | 4-08 | 0 | — | 0 | 42 | 0 | 23 |
| | 4-09 | 0 | — | 0 | 53 | 0 | 10 |
| | 4-11 | 0 | — | 0 | 40 | 0 | 0 |
| | 4-12 | 0 | — | 0 | 26 | 0 | 0 |
| | 4-13 | 0 | — | 0 | 22 | 0 | 9 |
| | 4-14 | 0 | — | 0 | 26 | 0 | 8 |
| | 4-15 | 0 | — | 0 | 47 | 0 | 9 |

Original tumor implanted on the right side. Re-challenged EMT6 implanted on the left side on study Day 70. Data assembled from measurements captured in WebDirector (S003617).

The data of Table 12 show that the combination treatment resulted in not only complete regression of tumors (CR), but also induced immune memory. Translation to clinical outcome is that the combination treatment may result in durable remission in patients.

### A Study of Anti-PD-L1 Checkpoint Antibody (LY3300054) Alone and in Combination in Participants with Advanced Refractory Solid Tumors (PACT); Study Treatment Arm D: LY3300054+Merestinib

Clinical Study Design

The clinical trial study, NCT02791334, is a multicenter Phase 1 study (hereinafter “the Study”), and Arm D is to assess the safety and tolerability of PD-L1 inhibitor LY3300054 both as a monotherapy and in combination with merestinib that may be safely administered to patients with advanced refractory solid tumors.

Study Objectives and Measures

The primary objectives of the Study in Phase 1 are to assess the safety and tolerability of PD-L1 inhibitor LY3300054, thereby identifying the recommended Phase 2 dose of LY3300054, administered as monotherapy and in combination with merestinib to participants with advanced solid tumors. The primary outcome measure is to identify the number of participants in the Study that display Dose-limiting toxicities (DLTs).

The secondary objectives of the Study in Phase 1 are to assess the pharmacokinetinetics in patients with solid tumors: (i) of PD-L1 inhibitor LY3300054 administered as monotherapy; (ii) of PD-L1 inhibitor LY3300054 administered in combination with merestinib; and (iii) of merestinib when administered in combination with LY3300054. The secondary outcome measures include, but are not limited to: (i) Minimum Concentration (Cₘᵢₙ) and approximate Maximum Concentration (Cₘₐₓ) of LY3300054 as monotherapy and when administered in combination with merestinib; (ii) Cₘᵢₙ and approximate Cₘₐₓ of merestinib when administered in combination with LY3300054; (iii) Objective Response Rate (ORR), as determined by RECIST criteria v. 1.1, to identify the proportion of participants with a Complete Response (CR) or Partial Response (PR): (iv) Progression Free Survival (PFS); (v) Duration of Response (DOR); (vi) Time-to-Repsonse (TTR); and (vii) Disease Control Rate (DCR).

SEQUENCE LISTING
Amino Acid and Nucleotide Sequences

SEQ ID NO: 1 (human PD-L1)
MRIFAVFIFMTYWHLLNAFTVTVPDLYVVEYGSNMTIECKFPVEKQLDLA
ALIVYWEMEDKNIIQFVHGEEDLKVQHSSYRQRARLLKDQLSLGNAALQI
TDVKLQDAGVYRCMISYGGADYKRITVKVNAPYNKINQRILVVDPVTSEH
ELTCQAEGYPKAEVIWTSSDHQVLSGKTTTTNSKREEKLFNVTSTLRINT
TTNEIFYCTFRRLDPEENHTAELVIPELPLAHPPNERTHLVILGAILLCL
GVALTFIFRLRKGRMMDVKKCGIQDTNSKKQSDTHLEET
SEQ ID NO: 2 (HCDR1 of LY3300054)
KASGGTFSSYAIS
SEQ ID NO: 3 (HCDR2 of LY3300054)
GIIPIFGTANYAQKFQG
SEQ ID NO: 4 (HCDR3 of LY3300054)
ARSPDYSPYWYGNIDV
SEQ ID NO: 5 (LCDR1 of LY3300054)
SGSSSNIGSNPVN
SEQ ID NO: 6 (LCDR2 of LY3300054)
YGNSNRPS
SEQ ID NO: 7 (LCDR3 of LY3300054)
QSYDSSLSGSV
SEQ ID NO: 8 (HCVR of LY3300054)
QVQLVQSGAEVKKPGSSVKVSCKASGGTFSSYAISWVRQAPGQGLEWMGG
IIPIFGTANYAQKFQGRVTITADKSTSTAYMELSSLRSEDTAVYYCARSP
DYSPYYYYGMDVWGQGTTVTVSS
SEQ ID NO: 9 (LCVR of LY3300054)
QSVLTQPPSASGTPGQRVTISCSGSSSNIGSNTVNWYQQLPGTAPKLLIY
GNSNRPSGVPDRFSGSKSGTSASLAISGLQSEDEADYYCQSYDSSLSGSV
FGGGIKLTVLG
SEQ ID NO: 10 (HC of LY3300054)
QVQLVQSGAEVKKPGSSVKVSCKASGGTFSSYAISWVRQAPGQGLEWMGG
IIPIFGTANYAQKFQGRVTITADKSTSTAYMELSSLRSEDTAVYYCARSP
DYSPYYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPEAEGAPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPSSIEKTISKAKGQPR
EPQVYTLPPSREEMIKNQVSLTCLVKGFYPSDIAVEWESNGQPENNYKTT
PPVLDSDGSFFLYSKLTVDKSRWQQGNVFSCSVMHEALHNHYTQKSLSLS
PGK
SEQ ID NO: 11 (LC of LY3300054)
QSVLTQPPSASGTPGQRVTISCSGSSSNIGSNTVNWYQQLPGTAPKLLIY
GNSNRPSGVPDRFSGSKSGTSASLAISGLQSEDEADYYCQSYDSSLSGSV
FGGGIKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPAECS
SEQ ID NO: 12 (DNA of HC of LY3300054)
CAGGTCCAGCTGGTCCAGTCAGGGGCCGAGGTCAAAAAGCCAGGGTCATC
TGTCAAAGTGTCTTGTAAGGCATCCGGGGGCACATTTTCCAGCTACGCTA
TCTCCTGGGTGAGACAGGCACCAGGGCAGGGTCTGGAGTGGATGGGCGGA
ATCATTCCCATCTTCGGGACCGCCAACTACGCTCAGAAGTTTCAGGGAAG
GGTCACTATTACCGCCGACAAAAGCACATCTACTGCTTATATGGAGCTGT
CTAGTCTGAGGTCTGAAGATACCGCAGTGTACTATTGCGCCCGGAGTCCC
GACTATAGCCCTTACTATTACTATGGCATGGATGTCTGGGGCCAGGGAAC
CACAGTGACAGTCTCATCCGCTAGCACCAAGGGCCCATCGGTCTTCCCCC
TGGCACCCTCCTCCAAGAGCACCTCTGGGGGCACAGCGGCCCTGGGCTGC
CTGGTCAAGGACTACTTCCCCGAACCGGTGACGGTGTCGTGGAACTCAGG
CGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGC
ACCCAGACCTACATCTGCAACGTGAATCACAAGCCCAGCAACACCAAGGT
GGACAAGAGAGTTGAGCCCAAATCTTGTGACAAAACTCACACATGCCCAC
CGTGCCCAGCACCTGAAGCCGAGGGGGCACCGTCAGTCTTCCTCTTCCCC
CCAAAACCCAAGGACACCCTCATGATCTCCCGGACCCCTGAGGTCACATG
GGTGGTGGTGGACGTGAGCCACGAAGACCCTGAGGTCAAGTTCAACTGGT
ATGTGGACGGCGTGGAGGTGCATAATGCCAAGACAAAGCCGCGGGAGGAG
CAGTACAACAGCACGTACCGTGTGGTCAGCGTCCTCACCGTCCTGCACCA
AGACTGGCTGAATGGCAAGGAGTACAAGTGCAAGGTCTCCAACAAAGCCC
TCCCATCCTCCATCGAGAAAACCATCTCCAAAGCCAAAGGGCAGCCCCGA
GAACCACAGGTGTACACCCTGCCCCCATCCCGGGAGGAGATGACCAAGAA
CCAAGTCAGCCTGACCTGCCTGGTCAAAGGCTTCTATCCCAGCGACATCG
CCGTGGAGTGGGAGAGCAATGGGCAGCCGGAGAACAACTACAAGACCACG
CCTCCCGTGCTGGACTCCGACGGCTCCTTCTTCCTCTATTCCAAGCTCAC
CGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCT
CCGGGCAAA
SEQ ID NO: 13 (DNA of LC of LY3300054)
CAGTCCGTCCTGACACAGCCACCCTCAGCCTCTGGCACCCCTGGGCAGCG
AGTGACAATCTCTTGTTCTGGGAGTTCCTCAAATATTGGTAGTAACACCG
TGAATTGGTACCAGCAGCTGCCCGGCACAGCACCTAAGCTGCTGATCTAT
GGAAACTCAAATAGGCCATCCGGAGTCCCCGACCGGTTCTCTGGTAGTAA
ATCAGGCACTTCCGCCAGCCTGGCTATTAGCGGGCTGCAGTCTGAGGACG
AAGCCGATTACTATTGCCAGTCTTACGATTCCAGCCTGTCTGGAAGTGTG
TTTGGCGGAGGGATCAAGCTGACCGTCCTGGGCCAGCCTAAGGCTGCCCC
CTCGGTCACTCTGTTCCCGCCCTCCTCTGAGGAGCTTCAAGCCAACAAGG
CCACACTGGTGTGTCTCATAAGTGACTTCTACCCGGGAGCCGTGACAGTG
GCCTGGAAGGCAGATAGCAGCCCCGTCAAGGCGGGAGTGGAGACCACCAC
ACCCTCCAAACAAAGCAACAACAAGTACGCGGCCAGCAGCTACCTGAGCC
TGACGCCTGAGCAGTGGAAGTCCCACAGAAGCTACAGCTGCCAGGTCACG
CATGAAGGGAGCACCGTGGAGAAGACAGTGGCCCCTGCAGAATGCTCT
SEQ ID NO: 14 (HC 178G7)
QVQLQQSGADLAKPGSSVKISCKASGYNFNSYYINWIKQTTGQGLEYIGY
INTVSGTTKYSEKFKGKATLTVDKSSSTAFMQLSSLTPDDSAVYYCARGT
IVLDDYWGQGVKVTVSSAETTAPSVYPLAPGTALKSNSMVTLGCLVKGYF
PEPVTVTWNSGALSSGVHTFPAVLQSGLYTLTSSVTVPSSTWPSQTVTCN
VAHPASSTKVDKKIVPRNCGGDCKPCICTGSEVSSVFIFPSKPKDVLTIT
LTPKVTCVVVDISQDDPEVHFSWFVDDVEVHTAQTRPPEEQFNSTFRSVS
ELPILHQDWLNGRTFRCKVTSAAFPSPIEKTISKPEGRTQVPHVYTMSPT
KEEMTQNEVSITCMVKGFYPPDIYVEWQMNGQPQENYKNTPPTMDTDGSY
FLYSKLNVKKEKWQQGNTFTCSVLHEGLHNHHTEKSLSHSPGK
SEQ ID NO: 15 (LC 178G7)
DIVMTQTPSSQAVSAGEKVTMSCKSSQSLLYNEKKKNYLAWYQQKPGQSP
KLLIYWASTRESGVPDRFLGSGSGTDFTLTINSVQAEDLAVYYCQQSYDF
PRTFGGGTKLELKRADAAPTVSIFPPSTEQLATGGASVVCLMNNFYPRDI
SVKWKIDGTERRDGVLDSVTDQDSKDSTYSMSSTLSLTKADYESHNLYTC
EVVHKTSSSPVVKSFNRNEC

1. A method of treating cancer, comprising administering to the patient an effective amount of merestinib, or a pharmaceutically acceptable salt thereof, in combination with an effective amount of an anti-human PD-L1 antibody or an anti-human PD-1 antibody,
wherein the cancer is lung cancer, non-small cell lung cancer, breast cancer, melanoma, colorectal cancer, pancreatic cancer, biliary tract cancer, melanoma, uveal melanoma, sarcoma, bladder cancer, renal cancer, urinary tract cancer, head cancer, neck cancer, thyroid cancer, ovarian cancer, hereditary papillary renal cell carcinoma, hepatocellular carcinoma, or gastric cancer.

2. The method of claim 1, wherein the anti-human PD-L1 antibody is atezolizumab, YW243.55.S70, MEDI-4736, MSB-0010718C, MDPL3280A, durvalumab, avelumab, or MDX-1105.

3. The method of claim 1, wherein the anti-human PD-L1 antibody is LY3300054.

4. The method of claim 1, wherein the anti-human PD-1 antibody is nivolumab, pembrolizumab, pidilizumab, or AMP-224.

5. The method of claim 1, wherein the cancer is breast cancer, melanoma, or colorectal cancer.

6. The method claim 1, wherein merestinib, or a pharmaceutically acceptable salt thereof, is administered at a dose of 40 mg to 120 mg once daily in a 21-day cycle or a 28-day cycle, and the anti-human PD-L1 antibody or the anti-human PD-1 antibody is administered at a dose of 1 mg/kg to 10 mg/kg on Day 1 of a 14-day cycle, on Day 1 of a 21-day cycle, on Day 1 and Day 8 of a 21-day cycle, on Day 1 and Day 15 of a 21-day cycle, on Day 1, Day 8, and Day 15 of a 21-day cycle, on Day 1 of a 28-day cycle, or on Day 1 and Day 15 of a 28-day cycle.

7. The method of claim 6, wherein merestinib, or a pharmaceutically acceptable salt thereof, is administered simultaneously with the anti-human PD-L1 antibody or an anti-human PD-1 antibody.

8.-14. (canceled)
